(19) United States
(12) Patent Application Publication
(10) Pub. No.: US 2006/0252032 A1
(43) Pub. Date: Nov. 9, 2006

(54) Detection of human herpesviruses

(75) Inventors: Azamat Aslanukov, Verona, WI (US); Victor Lyamichev, Madison, WI (US); Hatim Allawi, Madison, WI (US); Vecheslav Elagin, Waunakee, WI (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 11/342,459

(22) Filed: Jan. 30, 2006

Related U.S. Application Data
(60) Provisional application No. 60/648,137, filed on Jan. 28, 2005.

(51) Int. Cl. C12Q 1/70; C12Q 1/68

(52) U.S. Cl. 435/5; 435/6

(57) ABSTRACT

The present invention provides methods, compositions, and kits related to nucleic acid detection assays for detecting human herpes virus. For example, the present invention provides detection assays for detecting human herpes virus subtypes HHV1- through HHV-8.

The present Application claims priority to U.S. Provisional Application Ser. No. 60/648,137, filed Jan. 28, 2005, which is incorporated herein by reference in its entirety.

### FIELD OF THE INVENTION

The present invention provides methods, compositions and kits related to nucleic acid detection assays for detecting human herpes virus. For example, the present invention provides detection assays for detecting human herpes virus subtypes HHV1- through HHV-8.

### BACKGROUND

Certain methods for detecting Human Herpes virus are known in the art. General methods for amplification of Herpesvirus DNA by polymerase chain reaction (PCR) are described in U.S. Pat. No. 5,176,995 (herein incorporated by reference). Alternative methods for nucleic acid amplification have also been described for Herpesvirus DNA, including gap-filling ligase chain reaction (LCR), (e.g., U.S. Pat. No. 5,427,930, herein incorporated by reference). The LightCycler quantitative PCR method detects the products of nucleic acid amplification by neighboring hybridization of probes bearing fluorescent label. This assay is available for use with HSV-1, HSV-2, and EBV (Roche Diagnostics Corporation, F. Hoffinann-La Roche Ltd, Basel, Switzerland), (see e.g, US Pat. Pub. 2002/0164586, herein incorporated by reference).

The products of nucleic acid amplification or of the target nucleic acid itself may also be detected by electrophoretic gel separation and hybridization to a labeled oligonucleotide probe (e.g., U.S. Pat. No. 5,354,653 herein incorporated by reference). The products of nucleic acid amplification or of the target nucleic acid itself may also be detected by retardation of electrophoretic mobility through a gel in the presence of an oligonucleotide probe (e.g., U.S. Pat. No. 5,846,706 herein incorporated by reference). Further, the products of nucleic acid amplification or of the target nucleic acid itself may also be detected by restriction fragment length polymorphism (RFLP) analysis (e.g., U.S. Pat. No. 4,762,780 herein incorporated by reference).

The Hybrid Capture 2 (HC2) test relies on hybridization of target DNA to complimentary RNA probes. The resultant RNA-DNA hybrids are recognized by surface-bound antibodies as well as antibodies conjugated to alkaline phosphatase, allowing generation of a chemiluminescent signal in the presence of appropriate substrates (Digene, Gaithersburg, Md.). Such a test is available for the detection of CMV target nucleic acid.

Certain additional techniques have been described for the detection of Herpesvirus in a sample. For example, U.S. Pat. No. 6,110,677 (herein incorporated by reference), describes an assay in which a single oligonucleotide is reversibly hybridized to a target nucleic acid, and bound oligonucleotide is cleaved by a 5′nuclease. In this assay the product of the cleavage reaction accumulates over time under isothermal conditions, and is representative of the presence of the target nucleic acid in the sample under examination. The TAQMAN quantitative real-time PCR instrument can be used to monitor the products of nucleic acid amplification using dequenching of fluorescent label from a nucleic acid probe hybridized to the product of the nucleic acid amplification reaction. This assay has been applied to detect HSV-1, HSV-2, VZV, and CMV (e.g., U.S. Pat. No. 5,210,015 and van Doomum, et al, J. Clin. Microbiol., 41, pp. 576-580, 2003; both of which are herein incorporated by reference).

Each of the diagnostic techniques described above possess inherent limitations such as high rates of false positive and negative detection, limited quantitation dynamic range, high cost, long time periods for results, and requirements for large quantities of target material in the specimen. Therefore, there exists a need for a rapid, sensitive, and highly quantitative direct detection assay for detecting Herpesvirus infection in clinical samples.

### SUMMARY OF THE INVENTION

The present invention provides methods, compositions and kits related to nucleic acid detection assays for detecting human herpes virus. For example, the present invention provides detection assays for detecting human herpes virus subtypes HHV1- through HHV-8. In some preferred embodiments, a single oligonucleotide is used as a PCR primer and also serves as the INVADER oligonucleotide or other component of an invasive cleavage reaction.

In some embodiments, the present invention provides methods of detecting the presence or absence of at least one human herpes virus subtype in a sample comprising; a) contacting the sample with first and second oligonucleotides, wherein the first and second oligonucleotides are configured to form invasive cleavage structures with a target sequence, wherein the target sequence comprises human herpes virus sequences, and b) detecting the presence or absence of the at least one human herpes virus subtype in the sample.

In certain embodiments, the present invention provides kits for detecting the presence or absence of at least one human herpes virus subtype in a sample comprising; a) first and second oligonucleotides configured to form an invasive cleavage structure with a target sequence, wherein the target sequence comprises human herpes virus sequences, and b) a cleavage agent, wherein the cleavage agent is capable of cleaving the first oligonucleotide when the cleavage structure is formed.

In particular embodiments, the detecting comprises observing a signal generated by cleavage of the first oligonucleotide, thereby identifying the presence of the at least one human herpes virus subtype in the sample. In other embodiments, the at least one human herpes virus subtype is selected from the group consisting of: HHV-1, HHV-2, HHV-3, HHV-4, HHV-5, HHV-6, HHV-7, and HHV-8. In further embodiments, the human herpes virus sequences comprises at least a portion of a gene or sequence selected from the group consisting of: HHV-1 Thymidine Kinase gene (e.g., SEQ ID NO: 1 or SEQ ID NO:6); HHV-2 Tymidine Kinase gene (e.g. SEQ ID NO:2 or SEQ ID NO:16), HHV-3 gh or gB genes (e.g. SEQ ID NOs:21, 25, 29, or 33), HHV-4 BamHI-W region (e.g. SEQ ID NO:37 or 44), HHV-5 DNA polymerase gene (e.g. SEQ ID NO:50 or 56), HHV-6 U90 gene (e.g. SEQ ID NO:58 or 67), HHV-7 MYG gH gene (e.g. SEQ ID NO:71 or 75), HHV-8 glycoprotein K1 gene (e.g., SEQ ID NO:77 or 86) or any of the sequences shown in Table 1 or variants thereof. In additional embodiments, the method further comprises identifying one or more genotypes or serotypes within the at least one human herpes subtype.

In certain embodiments, the first oligonucleotide (e.g., primary probe) comprises a 5′ portion and a 3′ portion, wherein the 3′ portion is configured to hybridize to the target sequence, and wherein the 5′ portion is configured to not hybridize to the target sequence. In some embodiments, the second oligonucleotide (e.g., INVADER oligonucleotide) comprises a 5′ portion and a 3′ portion, wherein the 5′ portion is configured to hybridize to the target sequence, and wherein the 3′ portion is configured to not hybridize to the target sequence.

In certain embodiments, the second oligonucleotide is a multipurpose oligonucleotide as it also serves as a first PCR primer configured to PCR amplify the target sequence with a second PCR primer. Additional information on multipurpose oligonucleotides is found in U.S. Provisional Application 60/624,626, filed Nov. 3, 2004, which is herein incorporated by reference. In some embodiments, the second (multipurpose) oligonucleotide, which also serves as the first PCR primer, is selected from the group consisting of: SEQ ID NO:4, SEQ ID NO:8, SEQ ID NO:13,.SEQ ID NO:19, SEQ ID NO:22, SEQ ID NO:26, SEQ ID NO:30, SEQ ID NO:35, SEQ ID NO:39, SEQ ID NO:43, SEQ ID NO:47, SEQ ID NO:49, SEQ ID NO:54, SEQ ID NO:55, SEQ ID NO:61, SEQ ID NO:62, SEQ ID NO:63, SEQ ID NO:66, SEQ ID NO:70, SEQ ID NO:74, SEQ ID NO:80, SEQ ID NO:81, SEQ ID NO:85, or similar sequences such as any of the preceding sequences with one or more bases changed to a different base.

In particular embodiments, the methods further comprise contacting the sample with the second PCR primer (e.g. for a single reaction vessel detection assay containing both PCR primers, one of which also serves as the INVADER oligonucleotide, as well as the primary probe and any other INVADER assay reagents). In some embodiments, the second PCR primer is selected from the group consisting of: SEQ ID NO:3, SEQ ID NO:9, SEQ ID NO:11, SEQ ID NO:12, SEQ ID NO:17, SEQ ID NO:23, SEQ ID NO:27, SEQ ID NO:31, SEQ ID NO:34, SEQ ID NO:38, SEQ ID NO:40, SEQ ID NO:42, SEQ ID NO:48, SEQ ID NO:52, SEQ ID NO:53, SEQ ID NO:59, SEQ ID NO:60, SEQ ID NO:65, SEQ ID NO:69, SEQ ID NO:73, SEQ ID NO:78, SEQ ID NO:79, SEQ ID NO:84, or similar sequences such as any of the preceding sequences with one or more bases changed to a different base.

In additional embodiments, the first oligonucleotide is selected from the group consisting of: SEQ ID NO:5, SEQ ID NO:10, SEQ ID NO:14, SEQ ID NO:20, SEQ ID NO:24, SEQ ID NO:28, SEQ ID NO:32, SEQ ID NO:36, SEQ ID NO:41, SEQ ID NO:45, SEQ ID NO:46, SEQ ID NO:51, SEQ ID NO:57, SEQ ID NO:64, SEQ ID NO:68, SEQ ID NO:72, SEQ ID NO:76, SEQ ID NO:82, SEQ ID NO:83, and SEQ ID NO:87. In other embodiments, the methods further comprise contacting the sample with a FRET cassette. In some embodiments, the target sequence comprises an amplified product (e.g., PCR amplified product). In certain embodiments, the target sequence comprises genomic herpes virus nucleic acid.

In other embodiments, the first or second oligonucleotide comprises one or more mismatches with the target sequence. In particular embodiments, the first or second oligonucleotide do not hybridize to human genomic DNA under stringent conditions. In some embodiments, the first and second oligonucleotides are configured such that a stable duplex between the first and second oligonucleotides and the target sequence is not formed. In additional embodiments, the first and/or second oligonucleotides are not fully complementary to the target sequence.

In certain embodiments, the methods further comprise contacting the sample with third and fourth oligonucleotides configured to form a second invasive cleavage structure (e.g. for detecting a second human herpes virus subtype). In other embodiments, the sample is a biological sample (e.g. blood, urine sample, semen sample, biopsy, etc.). In certain embodiments, the sample is obtained from a subject suspected of being infected with a human herpes virus. In preferred embodiments, the human herpes virus subtype is detected without intervening purification or extraction steps or enrichments steps (e.g. a raw blood sample is contacted with the detection assay without purification, extraction or enrichment). In some embodiments, the detecting is performed on a microfluidic device (e.g. wherein centripetal force is applied to the microfluidic device). In certain preferred embodiments, the microfluidic devices such as those described in U.S. Pat. Nos. 6,627,159; 6,720,187; 6,734,401; and 6,814,935, as well as U.S. Pat. Pub. 2002/0064885, all of which are herein incorporated by reference.

The method is not limited by the nature of the target nucleic acid. In some embodiments, the target nucleic acid is single stranded or double stranded DNA or RNA. In some embodiments, double stranded nucleic acid is rendered single stranded (e.g., by heat) prior to formation of the cleavage structure. In some embodiments, the source of target nucleic acid comprises a sample containing genomic DNA. Samples include, but are not limited to, tissue sections, blood, saliva, cerebral spinal fluid, pleural fluid, milk, lymph, sputum and semen.

In some embodiments, the detected herpes sequences are any of those found below in Table I (GenBank accession numbers are provided) or variants thereof. It is understood that sequences will diverge over time and that other HHV varieties, now known, or later discovered are readily adaptable to the methods and composition of the present invention, per the description herein.

TABLE 1

| HHV-1 | HHV-2 | HHV-3 | | HHV-5 | HHV-6 | HHV-7 | HHV-8 |
|---|---|---|---|---|---|---|---|
| (e.g., Thymidine | (e.g. Thymidine | (e.g., gH | | (e.g., DNA | (e.g., U90 | (e.g. MYG | (e.g. glycoprotein |
| Kinase) | Kinase) | and gB) | HHV-4 | Polymerase) | gene) | gH) | K1 gene) |
| J04327 | X01712 | AY253697 | V01555 | AF133615 | AY245907 | AF343029 | AY735058 |
| V00467 | M29942 | AY253679 | AJ507799 | AF133617 | AY245908 | AF037218 | AY736219 |
| J02224 | M29941 | AY253709 | M80517 | AF133619 | AY245906 | AF343027 | AF133042 |
| L19900 | M29943 | AY253703 | M15972 | AY422367 | AF157706 | AF343023 | AY735054 |
| X14112 | M29940 | AY253691 | J02069 | AY422364 | AY245904 | AF343021 | AY736218 |
| V00470 | Z86099 | AY253673 | M15973 | AY422361 | AY245902 | AF343020 | AF130304 |
| AF057310 | V00466 | AY548171 | | AY422371 | AY245905 | U43400 | AF278834 |
| AB047358 | S46714 | AY548170 | | AY422373 | AY245903 | AF343022 | AF178806 |
| AB047377 | AY038369 | X04370 | | AF133627 | AY245913 | AF343028 | AF178803 |
| AB047374 | AB009262 | AY017043 | | AF133614 | AY245911 | AF343025 | AF178793 |
| AB047365 | AB009261 | AY010902 | | AF133623 | AY245910 | AF343026 | AF278823 |
| AB047378 | AB009256 | AY013747 | | AF133608 | AY245912 | AF007831 | AY735050 |
| AB047370 | AB178228 | AF325436 | | AF133591 | AY245909 | AF343019 | AY735043 |
| AB047375 | AB009263 | AY016462 | | AY304062 | U92288 | AF343024 | AY766083 |
| AB047373 | AB178230 | AY016457 | | AY304061 | AB021506 | AF343030 | AY766082 |
| AB047372 | AB178229 | AF314216 | | AY282792 | AB075777 | AY192544 | AY735053 |
| AB047368 | S63520 | AF322634 | | AY282791 | AB075775 | AY192547 | AY735051 |
| AB047369 | AB009257 | AY253720 | | AY304059 | AB075773 | AY192546 | AY735047 |
| AB047376 | AY038368 | AY253685 | | AY304060 | AY245900 | AY192545 | AY735046 |
| AB047366 | | | | AF133598 | AY245899 | AY192549 | AF130302 |
| | | | | AF133613 | AY245896 | AY192548 | AF130274 |
| | | | | AF133589 | AY245895 | | AF282215 |
| | | | | AF133606 | AY245892 | | AF196316 |
| | | | | AF133626 | AY245891 | | AF282215 |

### DEFINITIONS

To facilitate an understanding of the present invention, a number of terms and phrases are defined below:

As used herein, the terms “subject” and “patient” refer to any organisms including plants, microorganisms and animals (e.g., mammals such as dogs, cats, livestock, and humans).

As used herein, the term “INVADER assay reagents” refers to one or more reagents for detecting target sequences, said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In some embodiments, the oligonucleotides comprise first and second oligonucleotides, said first oligonucleotide comprising a 5′ portion complementary to a first region of the target nucleic acid and said second oligonucleotide comprising a 3′ portion and a 5′ portion, said 5′ portion complementary to a second region of the target nucleic acid downstream of and contiguous to the first portion. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid.

In some embodiments, INVADER assay reagents are configured to detect a target nucleic acid sequence comprising first and second non-contiguous single-stranded regions separated by an intervening region comprising a double-stranded region. In preferred embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components.”

In some embodiments, the INVADER assay reagents further comprise a third oligonucleotide complementary to a third portion of the target nucleic acid upstream of the first portion of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In still other embodiments, the INVADER assay reagents further comprise an ARRESTOR molecule (e.g., ARRESTOR oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In preferred embodiments, predispensed assay reagent components are predispensed and are provided in a reaction vessel (including but not limited to a reaction tube or a well, as in, e.g., a microtiter plate). In certain preferred embodiments, the INVADER assay reagents are provided in microfluidic devices such as those described in U.S. Pats. Nos. 6,627,159; 6,720,187; 6,734,401; and 6,814,935, as well as U.S. Pat. Pub. 2002/0064885, all of which are herein incorporated by reference. In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, software (e.g., for collecting and analyzing data), inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tₘ.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof. In some embodiments, oligonucleotides that form invasive cleavage structures are generated in a reaction (e.g., by extension of a primer in an enzymatic extension reaction).

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “cleavage structure” as used herein, refers to a structure that is formed by the interaction of at least one probe oligonucleotide and a target nucleic acid, forming a structure comprising a duplex, the resulting structure being cleavable by a cleavage means, including but not limited to an enzyme. The cleavage structure is a substrate for specific cleavage by the cleavage means in contrast to a nucleic acid molecule that is a substrate for non-specific cleavage by agents such as phosphodiesterases which cleave nucleic acid molecules without regard to secondary structure (i.e., no formation of a duplexed structure is required).

The term “cleavage means” or “cleavage agent” as used herein refers to any means that is capable of cleaving a cleavage structure, including but not limited to enzymes. “Structure-specific nucleases” or “structure-specific enzymes” are enzymes that recognize specific secondary structures in a nucleic molecule and cleave these structures. The cleavage means of the invention cleave a nucleic acid molecule in response to the formation of cleavage structures; it is not necessary that the cleavage means cleave the cleavage structure at any particular location within the cleavage structure.

The cleavage means may include nuclease activity provided from a variety of sources including the CLEAVASE enzymes, the FEN-1 endonucleases (including RAD2 and XPG proteins), Taq DNA polymerase and E. coli DNA polymerase I. The cleavage means may include enzymes having 5′ nuclease activity (e.g., Taq DNA polymerase (DNAP), E. coli DNA polymerase I). The cleavage means may also include modified DNA polymerases having 5′ nuclease activity but lacking synthetic activity. Examples of cleavage means suitable for use in the method and kits of the present invention are provided in U.S. Pat. Nos. 5,614,402; 5,795,763; 5,843,669; 6,090; PCT Appln. Nos WO 98/23774; WO 02/070755A2; and WO0190337A2, each of which is herein incorporated by reference it its entirety.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage means with a cleavage structure (i.e., the treatment of a cleavage structure with a cleavage means).

The term “target nucleic acid,” when used in reference to an invasive cleavage reaction, refers to a nucleic acid molecule containing a sequence that has at least partial complementarity with at least a probe oligonucleotide and may also have at least partial complementarity with an INVADER oligonucleotide. The target nucleic acid may comprise single- or double-stranded DNA or RNA.

The term “non-target cleavage product” refers to a product of a cleavage reaction that is not derived from the target nucleic acid. As discussed above, in the methods of the present invention, cleavage of the cleavage structure generally occurs within the probe oligonucleotide. The fragments of the probe oligonucleotide generated by this target nucleic acid-dependent cleavage are “non-target cleavage products.”

The term “probe oligonucleotide,” when used in reference to an invasive cleavage reaction, refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide—whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “cassette,” when used in reference to an invasive cleavage reaction, as used herein refers to an oligonucleotide or combination of oligonucleotides configured to generate a detectable signal in response to cleavage of a probe oligonucleotide in an INVADER assay. In preferred embodiments, the cassette hybridizes to a non-target cleavage product from cleavage of the probe oligonucieotide to form a second invasive cleavage structure, such that the cassette can then be cleaved.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label. In particularly preferred embodiments, cassette comprises labeled moieties that produce a fluorescence resonance energy transfer (FRET) effect.

As used herein, the phrase “non-amplified oligonucleotide detection assay” refers to a detection assay configured to detect the presence or absence of a particular polymorphism (e.g., SNP, repeat sequence, etc.) in a target sequence (e.g. genomic DNA) that has not been amplified (e.g. by PCR), without creating copies of the target sequence. A “non-amplified oligonucleotide detection assay” may, for example, amplify a signal used to indicate the presence or absence of a particular polymorphism in a target sequence, so long as the target sequence is not copied.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include comprise modified forms of deoxyribonucleotides as well as ribonucleotides.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

An oligonucleotide is said to be present in “excess” relative to another oligonucleotide (or target nucleic acid sequence) if that oligonucleotide is present at a higher molar concentration that the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present at least a 100-fold molar excess; typically at least 1 pmole of each probe oligonucleotide would be used when the target nucleic acid sequence was present at about 10 fmoles or less.

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

### DESCRIPTION OF THE DRAWINGS

FIG. 1 shows an exemplary HHV-1 (HSV-1) detection assay, where the target sequence is a portion of the HHV-1 Thymidine Kinase gene. FIG. 1 also shows an alignment of various HSV-1 variants such that conserved regions may be identified.

FIG. 2 shows another exemplary HHV-1 (HSV-1) detection assay, where the target sequence is a portion of the HHV-1 Thymidine Kinase gene.

FIG. 3 shows an exemplary HHV-2 (HSV-2) detection assay, where the target sequence is a portion of the HHV-2 Thymidine Kinase gene.

FIG. 4 shows another exemplary HHV-2 (HSV-2) detection assay, where the target sequence is a portion of the HHV-2 Thymidine Kinase gene.

FIG. 5 shows an exemplary HHV-3 (VZV) detection assay, where the target sequence is a portion of the HHV-3 gB gene.

FIG. 6 shows another exemplary HHV-3 (VZV) detection assay, where the target sequence is a portion of the HHV-3 gB gene.

FIG. 7 shows an exemplary HHV-3 (VZV) detection assay, where the target sequence is a portion of the HHV-3 gH gene.

FIG. 8 shows another exemplary HHV-3 (VZV) detection assay, where the target sequence is a portion of the HHV-3 gH gene.

FIG. 9 shows an exemplary HHV-4 (EBV) detection assay, where the target sequence is a portion of the HHV-4 BamHI-W region.

FIG. 10 shows another exemplary HHV-4 (EBV) detection assay, where the target sequence is a portion of the HHV-4 BamHI-W region.

FIG. 11 shows an exemplary HHV-5 (HCMV) detection assay, where the target sequence is a portion of the HHV-5 DNA polymerase gene.

FIG. 12 shows another exemplary HHV-5 (HCMV) detection assay, where the target sequence is a portion of the HHV-5 DNA polymerase gene.

FIG. 13 shows an exemplary HHV-6 detection assay, where the target sequence is a portion of the HHV-6 U90 gene.

FIG. 14 shows another exemplary HHV-6 detection assay, where the target sequence is a portion of the HHV-6 U90 gene.

FIG. 15 shows an exemplary HHV-7 detection assay, where the target sequence is a portion of the HHV-7 MYG gH gene.

FIG. 16 shows another exemplary HHV-7 detection assay, where the target sequence is a portion of the HHV-7 MYG gH gene.

FIG. 17 shows an exemplary HHV-8 (KSHV) detection assay, where the target sequence is a portion of the HHV-8 glycoprotein K1 gene.

FIG. 18 shows an exemplary HHV-8 (KSHV) detection assay, where the target sequence is a portion of the HHV-8 glycoprotein K1 gene.

FIG. 19 shows the results of using the detection assay shown in FIG. 11 to detect HCMV nucleic acid as described in Example 2.

FIG. 20 shows a schematic diagram of INVADER oligonucleotides, probe oligonucleotides and FRET cassettes for detecting a wild-type single-nucleotide polymorphism.

### DESCRIPTION OF THE INVENTION

The present invention provides methods, compositions and kits related to nucleic acid detection assays for detecting human herpes virus. For example, the present invention provides invasive cleavage assays, which may be combined with target amplification, for detecting human herpes virus subtypes HHV1- through HHV-8. In some preferred embodiments, a single oligonucleotide is used as a PCR primer and also serves as the INVADER oligonucleotide or other component of an invasive cleavage reaction.

I. Invasive Cleavage Assays

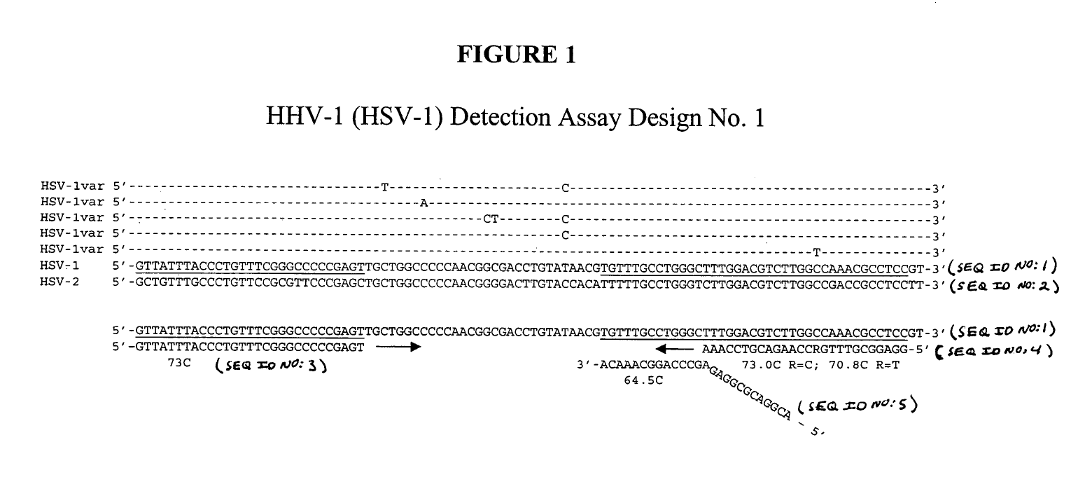

The present invention provides means for forming a nucleic acid cleavage structure that is dependent upon the presence of a target nucleic acid (e.g. comprising human herpes virus sequence such as shown in Table 1 and FIGS. 1-18) and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced. Such embodiments have been termed the INVADER assay (Third Wave Technologies) and are described in U.S. Pat. Nos. 5,846,717, 5,985,557, 5,994,069, 6,001,567, and 6,090,543, WO 97/27214 WO 98/42873, Lyamichev et al., Nat. Biotech., 17:292 (1999), Hall et al., PNAS, USA, 97:8272 (2000), each of which is herein incorporated by reference in their entirety for all purposes). The INVADER assay detects hybridization of probes to a target by enzymatic cleavage of specific structures by structure specific enzymes.

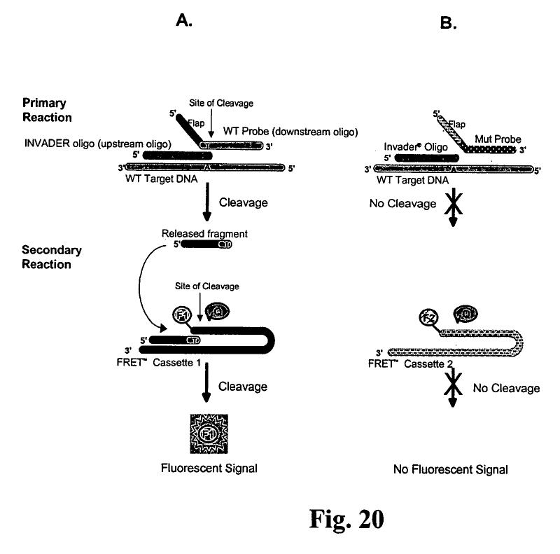

The INVADER assay detects specific DNA and RNA sequences by using structure-specific enzymes (e.g. FEN endonucleases) to cleave a complex formed by the hybridization of overlapping oligonucleotide probes (See, e.g. FIG. 20). Elevated temperature and an excess of one of the probes enable multiple probes to be cleaved for each target sequence present without temperature cycling. In some embodiments, these cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified, as well as amplified, RNA and DNA including genomic DNA. In the embodiments shown schematically in FIG. 20, the INVADER assay uses two cascading steps (a primary and a secondary reaction) both to generate and then to amplify the target-specific signal. For convenience, the alleles in the following discussion are described as wild-type (WT) and mutant (MT), even though this terminology does not apply to all genetic variations. In the primary reaction (FIG. 20, panel A), the WT primary probe and the INVADER oligonucleotide hybridize in tandem to the target nucleic acid to form an overlapping structure. An unpaired “flap” is included on the 5′ end of the WT primary probe. A structure-specific enzyme (e.g. the CLEAVASE enzyme, Third Wave Technologies) recognizes the overlap and cleaves off the unpaired flap, releasing it as a target-specific product. In the secondary reaction, this cleaved product serves as an INVADER oligonucleotide on the WT fluorescence resonance energy transfer (WT-FRET) probe to again create the structure recognized by the structure specific enzyme (panel A). When the two dyes on a single FRET probe are separated by cleavage (indicated by the arrow in FIG. 20), a detectable fluorescent signal above background fluorescence is produced. Consequently, cleavage of this second structure results in an increase in fluorescence, indicating the presence of the WT allele (or mutant allele if the assay is configured for the mutant allele to generate the detectable signal). In preferred embodiments, FRET probes having different labels (e.g. resolvable by difference in emission or excitation wavelengths, or resolvable by time-resolved fluorescence detection) are provided for each allele or locus to be detected, such that the different alleles or loci can be detected in a single reaction. In such embodiments, the primary probe sets and the different FRET probes may be combined in a single assay, allowing comparison of the signals from each allele or locus in the same sample.

If the primary probe oligonucleotide and the target nucleotide sequence do not match perfectly at the cleavage site (e.g., as with the MT primary probe and the WT target, FIG. 20, panel B), the overlapped structure does not form and cleavage is suppressed. The structure specific enzyme (e.g., CLEAVASE VIII enzyme, Third Wave Technologies) used cleaves the overlapped structure more efficiently (e.g. at least 340-fold) than the non-overlapping structure, allowing excellent discrimination of the alleles.

In the INVADER assays, the probes turn can over without temperature cycling to produce many signals per target (i.e., linear signal amplification). Similarly, each target-specific product can enable the cleavage of many FRET probes. The primary INVADER assay reaction is directed against the target DNA (or RNA) being detected. The target DNA is the limiting component in the first invasive cleavage, since the INVADER and primary probe are supplied in molar excess. In the second invasive cleavage, it is the released flap that is limiting. When these two cleavage reactions are performed sequentially, the fluorescence signal from the composite reaction accumulates linearly with respect to the target DNA amount.

In certain embodiments, the INVADER assay, or other nucleotide detection assays, are performed with accessible site designed oligonucleotides and/or bridging oligonucleotides. Such methods, procedures and compositions are described in U.S. Pat. No. 6,194,149, WO9850403, and WO198537, all of which are specifically incorporated by reference in their entireties. In certain embodiments, the target nucleic acid sequences are amplified prior to detection (e.g. such that amplified products are generated). In some embodiments, the target nucleic acid comprises genomic DNA. In other embodiments, the target nucleic acid comprises synthetic DNA or RNA. In some preferred embodiments, synthetic DNA within a sample is created using a purified polymerase. In some preferred embodiments, creation of synthetic DNA using a purified polymerase comprises the use of PCR. In other preferred embodiments, creation of synthetic DNA using a purified DNA polymerase, suitable for use with the methods of the present invention, comprises use of rolling circle amplification, (e.g., as in U.S. Pat. Nos. 6,210,884, 6,183,960 and 6,235,502, herein incorporated by reference in their entireties). In other preferred embodiments, creation of synthetic DNA comprises copying genomic DNA by priming from a plurality of sites on a genomic DNA sample. In some embodiments, priming from a plurality of sites on a genomic DNA sample comprises using short (e.g., fewer than about 8 nucleotides) oligonucleotide primers. In other embodiments, priming from a plurality of sites on a genomic DNA comprises extension of 3′ ends in nicked, double-stranded genomic DNA (i.e., where a 3′ hydroxyl group has been made available for extension by breakage or cleavage of one strand of a double stranded region of DNA). Some examples of making synthetic DNA using a purified polymerase on nicked genomic DNAs, suitable for use with the methods and compositions of the present invention, are provided in U.S. Pat. No. 6,117,634, issued Sep. 12, 2000, and U.S. Pat. No. 6,197,557, issued Mar. 6, 2001, and in PCT application WO 98/39485, each incorporated by reference herein in their entireties for all purposes. In some embodiments, the present invention provides methods for detecting a target sequence, comprising: providing a) a sample containing DNA amplified by extension of 3′ ends in nicked double-stranded genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequences and said oligonucleotides if said target sequences are present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

In other embodiments, synthetic DNA suitable for use with the methods and compositions of the present invention is made using a purified polymerase on multiply-primed genomic DNA, as provided, e.g., in U.S. Pat. Nos. 6,291,187, and 6,323,009, and in PCT applications WO 01/88190 and WO 02/00934, each herein incorporated by reference in their entireties for all purposes. In these embodiments, amplification of DNA such as genomic DNA is accomplished using a DNA polymerase, such as the highly processive Φ29 polymerase (as described, e.g., in U.S. Pat. Nos. 5,198,543 and 5,001,050, each herein incorporated by reference in their entireties for all purposes) in combination with exonuclease-resistant random primers, such as hexamers.

In some embodiments, the present invention provides methods for detecting a target sequence, comprising: providing a) a sample containing DNA amplified by extension of multiple primers on genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some preferred embodiments, said primers are random primers. In particularly preferred embodiments, said primers are exonuclease resistant. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequence and said oligonucleotides if said target sequence is present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

The present invention further provides assays in which the target nucleic acid is reused or recycled during multiple rounds of hybridization with oligonucleotide probes and cleavage of the probes without the need to use temperature cycling (e.g., for periodic denaturation of target nucleic acid strands) or nucleic acid synthesis (e.g., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (The kinetics of oligonucleotide replacement. Luis P. Reynaldo, Alexander V. Vologodskii, Bruce P. Neri and Victor I. Lyamichev. J. Mol. Biol. 97: 511-520 (2000)), multiple probes can hybridize to the same target, allowing multiple cleavages, and the generation of multiple cleavage products.

II. Human Herpes Viruses

Herpesviruses are complex viruses having a large genome consisting of double-stranded DNA encoding about 35 viral proteins. Herpesviruses encode a variety of enzymes involved in nucleic acid metabolism, DNA synthesis and protein processing. The Herpesviruses are widely separated in terms of genomic sequence and proteins, but all are similar in terms of virion structure and genome organization. The virion particles are about 180-200 nm in size, enveloped by a lipid bilayer, contain an icosahedral capsid, possess a linear double-stranded genome of about 130-230 kb in length, and virus replication and new particle assembly occurs in the host cell nucleus. More than 100 herpesviruses have been isolated. There are 8 known human Herpesviruses (HHV). These 8 virus types are further subdivided into 3 sub-families: The Alphaherpesviruses comprise the Herpes Simplex Viruses HSV-1 and HSV-2 (also known as HHV-1 and HHV-2) and Varicella-Zoster Virus (VZV), (also known as HHV-3). The Betaherpesviruses comprise Human Cytomegalovirus (HCMV), (also known as HHV-5) and Human Herpesviruses 6 (HHV-6) and 7 (HHV-7). The Gammaherpesviruses comprise Epstein-Barr Virus (EBV), (also known as HHV-4) and Kaposi Sarcoma Herpesvirus (KSHV), (also known as HHV-8).

The pathogenesis of the different Herpesviruses is varied. In the case of the Herpes Simplex Viruses, primary infection occurs through a break in the mucus membranes of the mouth or throat, via the eye or genitals or directly via minor abrasions in the skin. Because of the universal distribution of the virus, most individuals are infected by 1-2 years of age. Initial infection is usually asymptomatic, although there may be minor local vesicular lesions. Local multiplication ensues, followed by viremia and systemic infection. There then follows life-long latent infection with periodic reactivation. During primary infection, the virus enters peripheral sensory nerves and migrates along axons to sensory nerve ganglia in the CNS. During latent infection of nerve cells, viral DNA is maintained as an episome (not integrated) with limited expression of specific virus genes required for the maintenance of latency. The delicate balance of latency may be upset by various disturbances, physical (e.g., injury, U.V., hormones, etc) or psychological (stress, emotional disturbances, etc.). Reactivation of latent virus leads to recurrent disease—virus travels back down sensory nerves to the surface of body and replicates, causing tissue damage. HHV-1 (HSV-1) is primarily associated with oral and ocular lesions, while HHV-2 (HSV-2) is primarily associated with genital and anal lesions. Although painful, most recurrent infections resolve spontaneously, usually to reoccur later. More serious are herpetic keratitis (ulceration of cornea due to repeated infection which can lead to blindness) and encephalitis (very rare but often fatal). Incidence of genital herpes has increased sharply during the last few decades.

In the case of HHV-3 (VZV), infection normally gives rise to 2 distinct clinical syndromes: Varicella, commonly known as Chicken Pox, and Zoster, commonly known as Shingles. In the case of chicken pox, infection normally occurs in childhood via respiratory tract or conjunctiva. After multiplication at the inoculation sites, virus spreads to bloodstream and reticuloendothelial system. Secondary multiplication involves skin and mucosa, producing vesicles filled with very high titers of infectious virus. Complications are rare, but may include CNS infection. In the case of shingles, after primary infection, virus persists in sensory ganglia of CNS. It is not clear if this is a latent or a persistent infection, but ‘reactivation’ after many years leads to infection and tissue damage to dermatosome served by infected ganglia—most serious when cranial nerves are involved, affecting face and/or head which can lead to blindness. The complete sequence of the VZV genome is known, and is approximately 125 kbp in length.

In the case of HHV-4 (EBV), there exists a dual cell tropism for human B-lymphocytes (generally non-productive infection) and epithelial cells (productive infection). There is no suitable animal host, but replication/latency has been studied extensively in transformed human cell lines. Widespread worldwide, most populations have over a 90% infection rate. The usual outcome of infection is polyclonal B-cell activation and benign proliferation, which may be sub-clinical or produce infectious mononucleosis (glandular fever). Uniquely among Herpesviruses, there is a well-established relationship between EBV and oncogenesis—Burkitt's lymphoma and nasopharyngeal carcinoma. The complete nucleotide sequence (˜172 kbp) is known and contains many internal repeats.

In the case of HHV-5 (HCMV), the kinetics of CMV infection are ‘slow’—7-14 days compared to 24-48 h for HSV. As with some other Herpesviruses, certain parts of the CMV genome have considerable homology with cellular DNA, implying that the virus has acquired cellular genes during evolution. The complete nucleotide sequence is known and expression has been studied in detail. Upstream of the IE genes, there is a promoter/enhancer region which has been characterized in detail and is remarkable for its strength, which is why it is often used for heterologous expression of recombinant genes. This is the first region to be transcribed after infection and initiates replication. CMV infection is common (e.g. 60% of the UK population have experienced infection by the age of 40), with most infections being asymptomatic. Apart from during pregnancy and newborn infants exposed in utero, active (as opposed to latent) CMV infection only occurs in people with immune defects, specifically T-cell defects, such as AIDS patients and immunosuppressed transplant patients. Transmission is believed to be by an oral/respiratory route. Infection produces enlargement of cells and nuclear inclusion bodies in a wide range of tissues. In spite of the widespread distribution, CMV-related illness is rare and generally only occurs in two groups, immunocompromised and fetal infections. Evidence that the host immune response (particularly cell-mediated) plays a role in latency comes from the evidence of what occurs on immunosuppression. Latent virus is reactivated and AIDS/transplant patients experience frequent and severe infections with the potential for involvement of many possible organs. Fetal infections, particularly a problem when primary infection of the mother occurs, resulting in congenital abnormalities in a proportion of cases.

In the case of HHV-6, the virus was first isolated in 1986 in lymphocytes of patients with lymphoreticular disorders. The HHV-6 genome is about 160 kbp. HHV-6 is now recognized as being a universal human infection. Discovery of the virus solved a longstanding mystery—primary infection in childhood causes “roseola infantum” a.k.a. “fourth disease,” a common childhood rash whose cause was previously unknown. Antibody titers are highest in children and decline with age. Consequences of childhood infection appear to be mild. Primary infections of adults are rare but have more severe consequences—mononucleosis/hepatitis. HHV-6 infection is a problem in immunocompromised patients.

In the case of HHV-7, the virus was first isolated from human CD4+ cells in 1990. The HHV-7 genome is about 170 kbp, with an organization similar to but distinct from HHV-6. The complete genome sequence of HHV-7 has been determined and this shows a high degree of conservation of genetic content and encoded amino acid sequences to HHV-6. However, there is only limited antigenic cross-reactivity between the two viruses. At present, there is no clear evidence for the direct involvement of HHV-7 in any human disease, but HHV-7 might be a co-factor in HHV-6-related syndromes.

In the case of HHV-8 (KSHV), sequences of a unique herpesvirus were identified in 100% of amplifiable samples from AIDS patients with Kaposi's sarcoma (KS) and 15% of non-KS tissue samples from AIDS patients. There is a strong correlation (>95%) with KS in HIV+ and HIV− patients. The virus can be isolated from PBMC as well as KS tumor cells. Some evidence suggests that one of the genes of HHV-8, vGPCR (viral G-protein coupled receptor) acts as a vascular switch, turning on synthesis of a powerful angiogenic agent, vascular endothelial growth factor (VEGF), which is responsible for the development of KS. However, HHV-8 also contains a considerable number of other ‘pirated’ cellular genes in an ‘oncogenic cluster’ within the virus genome which may also be involved in the development of malignancy (See Boshoff, Nature 391: 24-25, 1998; and Nature Med. 4:435 1998). In addition to KS, this virus may also cause other tumors such as B-cell lymphomas (±EBV as ‘helper’). HHV-8 resembles EBV in that it has a tropism for epithelial and B-cells, and it is kept under immunological control and only presents a problem during immunosuppression.

III. Detection of Human Herpes Viruses

The present invention provides methods for detecting human herpes virus, including INVADER detection methods, either alone or simultaneous to or following a target amplification method (e.g. PCR). An exemplary embodiment of the detection methods of the present invention is as follows.

Material such as blood, saliva, semen, or other bodily fluid or tissue are collected from a specimen such as a human or animal patient of interest. Following this collection, the material is subsequently be treated further to extract RNA or DNA for later analysis. Cells of a certain desired type are separated from the specimen material prior to nucleic acid extraction. Alternatively, the specimen material nucleic acid is subjected directly to analysis by an invasive cleavage assay without any intervening sample preparation steps such as purification, extraction, enrichment, or the like.

Once the specimen material or purified nucleic acid is in its desired state for analysis by the invasive cleavage assay, one or more intervening steps may be employed to convert the nucleic acid to another desired form, or to produce additional copies of the target nucleic acid through any one of a number of procedures for nucleic acid amplification commonly known in the art. For example, the specimen RNA is converted by the action of a reverse transcriptase enzyme into a population of complimentary DNA molecules. In addition, or as an alternative, these reverse transcribed DNA molecules, or DNA derived directly from the specimen material, is amplified exponentially by the polymerase chain reaction (PCR).

Following these steps, the target nucleic acid is subjected to an invasive cleavage assay. In this assay, two oligonucleotides are configured to form an invasive.cleavage structure upon the target nucleic acid. Following formation of this invasive cleavage structure, a portion of one of the oligonucleotides forming the invasive cleavage structure is cleaved and released by an enzyme such as a Cleavase which recognizes this structure as a substrate. This cleavage of one of the oligonucleotides forming the invasive cleavage structure indicatives the presence of the target herpes nucleic acid in the sample. The presence of this cleavage product is assayed by any number of methods, including the presence of a labeled third oligonucleotide that will form another invasive cleavage structure with the product of the first cleavage reaction. The subsequent cleavage of this newly formed invasive cleavage structure is configured such that it results in the release of a detectable label. In such an assay, the presence of this released label indirectly indicates the presence of the target herpes nucleic acid in the specimen material of interest.

Various alternative configurations of such an experimental assay are readily employed. For example, one could employ a “multiplex” reaction, where several sets of invasive cleavage structure forming oligonucleotides are simultaneously added to the specimen material to simultaneously confirm the presence or absence of multiple target herpes nucleic acid sequences (e.g. HHV-1 and HHV-4), using any methodology that is able to differentiate the production of each of the products from each of the multiple cleavage reactions. As another example, it may be desirable to determine the presence or absence of multiple viruses of distinct phylogeny in the same sample, such as the simultaneous detection of a Herpesvirus and a Papillomavirus. In addition, or alternatively, it may be desirable to simultaneously determine the presence of an internal control nucleic acid that is expected to be present in a constant amount in each specimen material tested irregardless of the presence or absence of the target Herpesvirus nucleic acid. Additionally, such an experimental assay system may be modified to provide qualitative or quantitative information about the target nucleic acid in the sample. By comparison with an externally quantitated control nucleic acid molecule, tested in parallel in the same experimental assay, the quantity of the target Herpesvirus nucleic acid, if present in the specimen material, is determined.

### EXAMPLES

The following examples are provided in order to demonstrate and further illustrate certain preferred embodiments and aspects of the present invention and are not to be construed as limiting the scope thereof.

### Example 1

### Designing Herpes Virus Detection Assays

This example describes an exemplary process used to design detection assays to detect human herpes virus. In particular, this example describes the construction of PCR+INVADER detection assays for human herpes virus subtypes HHV-1 through HHV-8.

To begin designing detection assays for subtypes HHV-1-HHV-8, a particular gene from each of these subtypes was selected. Genes were selected based on the prevalence of sequence information available in the NCBI database such that conserved regions within these genes could be identified and employed in designing the assays. In this example, the following genes were selected for each of the subtypes: HHV-1—Thymidine Kinase; HHV-2—Tymidine Kinase; HHV-3—gH and gB; HHV-4—BamHI-W region (nt 33505-36576); HHV-5—DNA polymerase; HHV-6—U90 gene; HHV-7—MYG gH gene; and HHV-8—glycoprotein K1 gene.

Within each of the selected genes, the particular target sequence to be PCR amplified and then detected by the INVADER assay was selected using a set of five criteria. The five criteria used in this example is as follows:

1. The PCR amplicon region is selected such that it is generally less than 100 bases long.

2. The target region in the INVADER assay Primary Probe region is selected such that it is conserved among the many strains deposited in the NCBI database. If possible, the PCR primer regions are also selected such that they are conserved among the many strains. If a design that is conserved in the Primary Probe region cannot be found in a selected gene, then any base variations should be outside positions +1, +2, +3, +7, +8, and +9 relative to the cleavage site of the probe. For the INVADER oligo region, preferably, variability is only permitted beyond four bases from the cleavage site (i.e. -5, −6, −7, etc.). It is also preferable to avoid base variability towards the last 3-4 bases of the 3′ end of the forward primer.

3. The Primary Probe's melting temperature is selected to be about 63° C. The Primary Probe-target hybridization region is preferably selected to be less than 20 bases in length. The Primary Probe is also preferably selected such that it avoids forming stable unimolecular or biomolecular structures (e.g. hairpins or dimers).

4. The PCR primers and INVADER olio are preferably selected such that their melting temperature is greater than 65° C.

5. The Primary Probe and PCR primers are selected to avoid high homology to human DNA or other viral DNA (e.g. primer sequences are Blasted against NCBI's web site to ensure highly homologous sequences are not employed).

The five criteria can be used to design the PCR +INVADER herpes virus detection assays. It is noted that conserved regions within the selected genes can be identified using the sequence information at NCBI. For example, once a suitable region (e.g. about 350 bases in length) within one of these genes that fits many of the criteria listed above is identified, one can determine subregions that are conserved among all the serotypes deposited in NCBI's database by comparing among all of the deposited sequences. The top of FIGS. 1-4 and 13 show exemplary alignments that could be generated and used to determine the location of conserved regions.

Using the above criteria and methods, PCR+INVADER detections assays were designed for herpes subtypes HHV-1 through HHV-8. These assays designs are shown in FIGS. 1-18. For nearly all of these assay designs, one of the PCR primers also served as the INVADER oligonucleotide (e.g. to simplify assay design and efficiency). For each subtype, at least two detection assays are designed. Additional designs for each sub-type could be generated using the criteria outlined above (e.g. to target other regions in the sequences shown in Table 1).

FIG. 1 shows a detection assay design for detecting HHV-1 (also known as HSV-1). In this Figure, SEQ ID NO:1 is a selected portion of the HHV-1 Thymidine Kinase gene, a portion of which is to be PCR amplified, SEQ ID NO:3 is a forward primer (Tm=73C), SEQ ID NO:4 is the reverse primer (Tm=73C when R═C; Tm=70.8C when R=T) and also doubles as the INVADER oligonucleotide, and SEQ ID NO:5 is the primary probe (Tm=64.5C). This figure also shows an alignment of some of the HHV-1 variants, visually depicting how one could select conserved regions for the primary probe, as well as the PCR primers. It is noted that the “T” variation (rather than “C”) encompassed by the SEQ ID NO:4 reverse primer is accounted for by employing two different primers (i.e. in SEQ ID NO:4, “R” can be a “T” or a “C”).

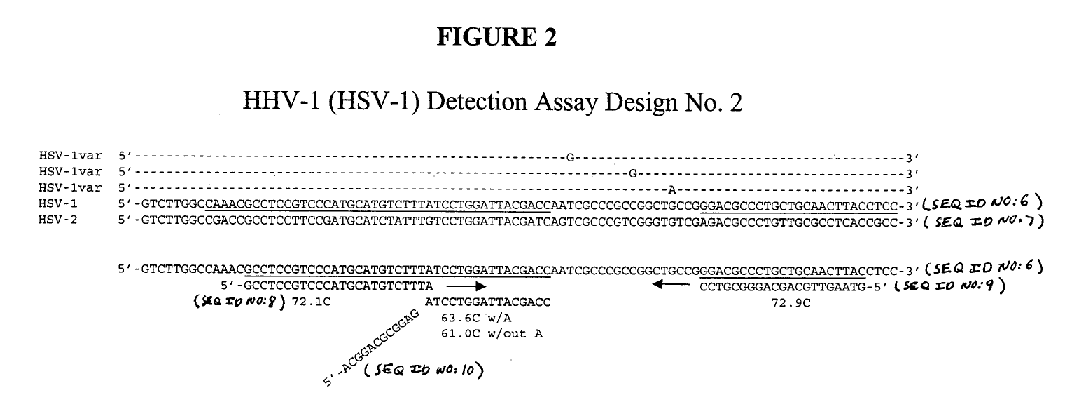

FIG. 2 shows an additional detection assay design for detecting HHV-1. In this Figure, SEQ ID NO:6 is a selected portion of the HHV-1 Thymidine Kinase gene, a portion of which is to be PCR amplified, SEQ ID NO:9 is a reverse primer (Tm=72.9C), SEQ ID NO:8 is a forward primer (Tm=72.1C) and also doubles at the INVADER oligonucleotide, and SEQ ID NO:10 is the primary probe (Tm=63.6C w/A, and 61.0C w/out A). This figure also shows an alignment of some of the HHV-1 variants, visually depicting how one could select conserved regions for the primary probe, as well as the PCR primers.

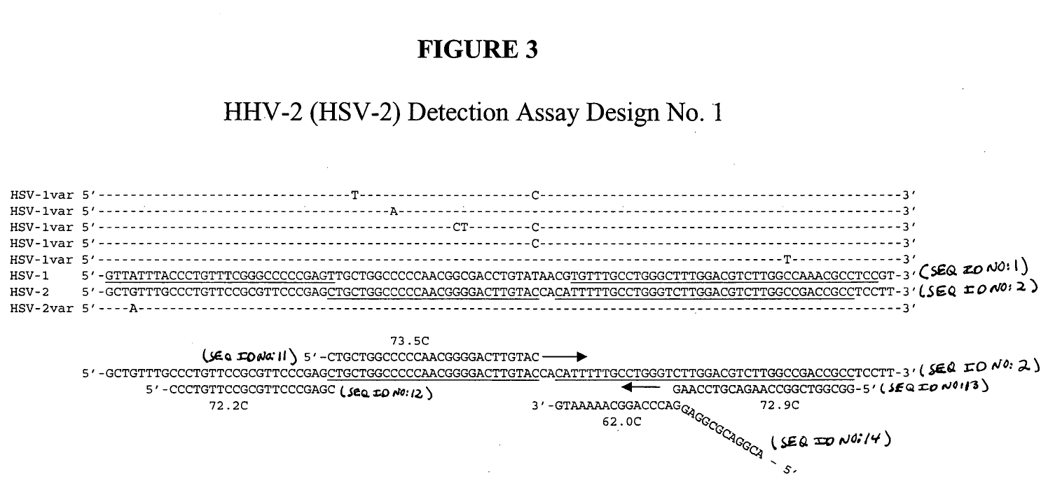

FIG. 3 shows a detection assay design for detecting HHV-2 (also known as HSV-2). In this Figure, SEQ ID NO:2 is a selected portion of the HHV-2 Thymidine Kinase gene, a portion of which is to be PCR amplified, SEQ ID NOs:11 and 12 are forward primers (Tm=73.5C and 72.2 respectively), SEQ ID NO: 13 is a reverse primer (Tm=72.9C) and also doubles as the INVADER oligonucleotide, and SEQ ID NO:14 is the primary probe (Tm=62.0C).

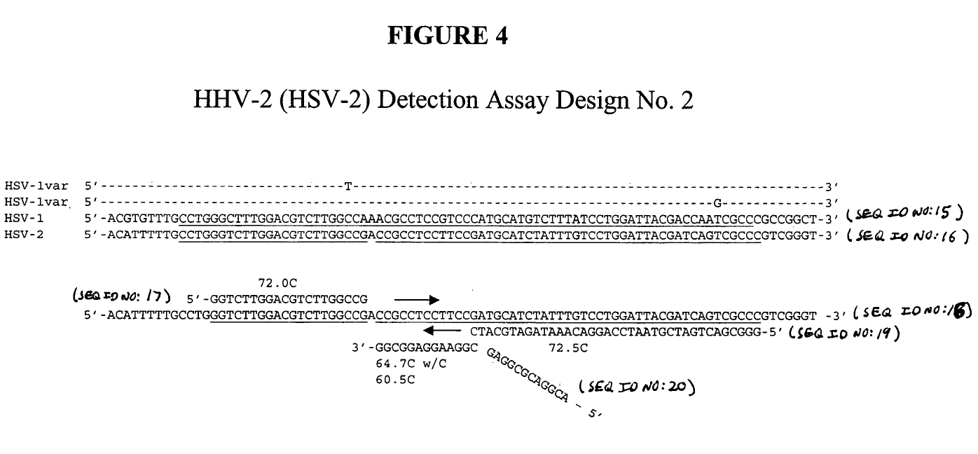

FIG. 4 shows an additional detection assay design for detecting HHV-2. In this Figure, SEQ ID NO:16 is a selected portion of the HHV-2 Thymidine Kinase gene, a portion of which is to be PCR amplified, SEQ ID NO:17 is a forward primer (Tm=72.0C), SEQ ID NO:19 is a reverse primer (Tm=72.5C) and also doubles as the INVADER oligonucleotide, and SEQ ID NO:20 is the primary probe (Tm=64.7C w/C; 60.5 w/out C).

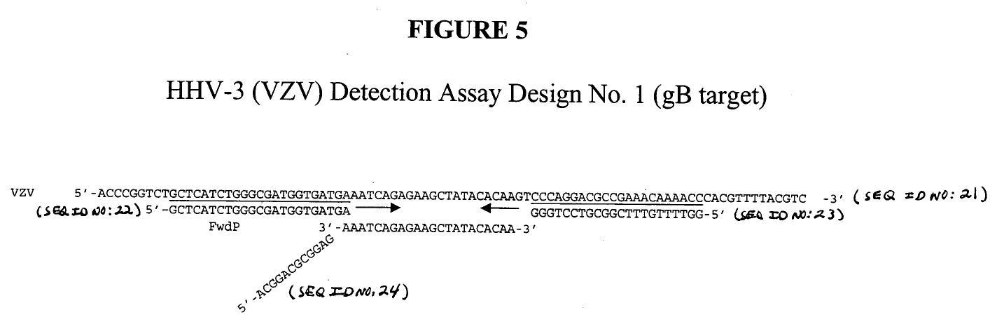

FIG. 5 shows a detection assay design for detecting HHV-3 (also known as Varicella-Zoster Virus or VZV). In this Figure, SEQ ID NO:21 is a selected portion of the HHV-3 gB gene, a portion of which is to be PCR amplified, SEQ ID NO:22 is a forward primer that also doubles as the INVADER oligonucleotide, SEQ ID NO:23 is a reverse primer, and SEQ ID NO:24 is the primary probe.

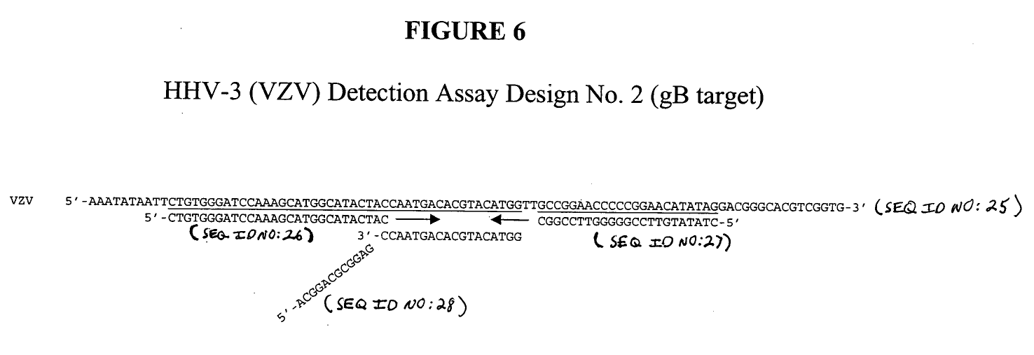

FIG. 6 shows an additional detection assay design for detecting HHV-3. In this Figure, SEQ ID NO:25 is a selected portion of the HHV-3 gB gene, a portion of which is to be PCR amplified, SEQ ID NO:26 is a forward primer which also double as the INVADER oligonucleotide, SEQ ID NO:27 is a reverse primer, and SEQ ID NO:28 is the primary probe.

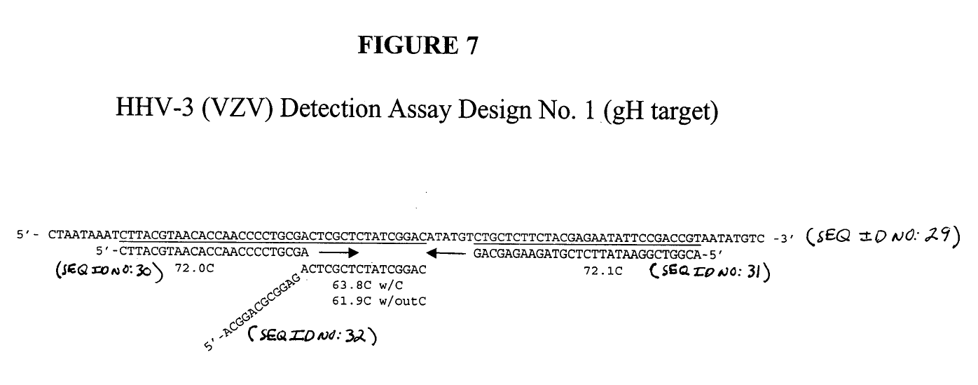

FIG. 7 shows an additional detection assay design for detecting HHV-3. In this Figure, SEQ ID NO:29 is a selected portion of the HHV-3 gH gene, a portion of which is to be PCR amplified, SEQ ID NO:30 is a forward primer which also double as the INVADER oligonucleotide, SEQ ID NO:31 is a reverse primer, and SEQ ID NO:28 is the primary probe.

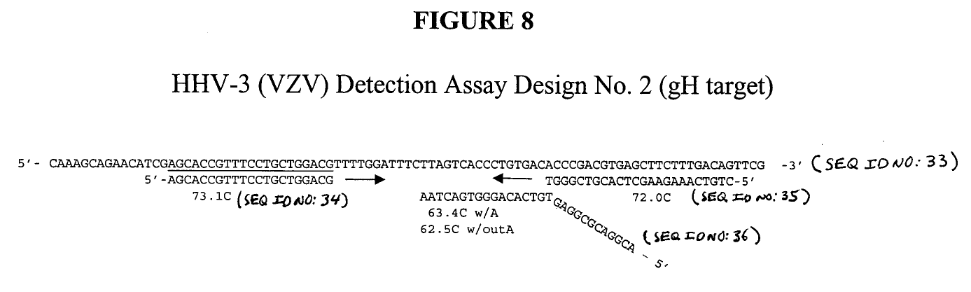

FIG. 8 shows an additional detection assay design for detecting HHV-3. In this Figure, SEQ ID NO:33 is a selected portion of the HHV-3 gH gene, a portion of which is to be PCR amplified, SEQ ID NO:34 is a forward primer, SEQ ID NO:35 is a reverse primer which doubles as an INVADER oligonucleotide, and SEQ ID NO:36 is the primary probe.

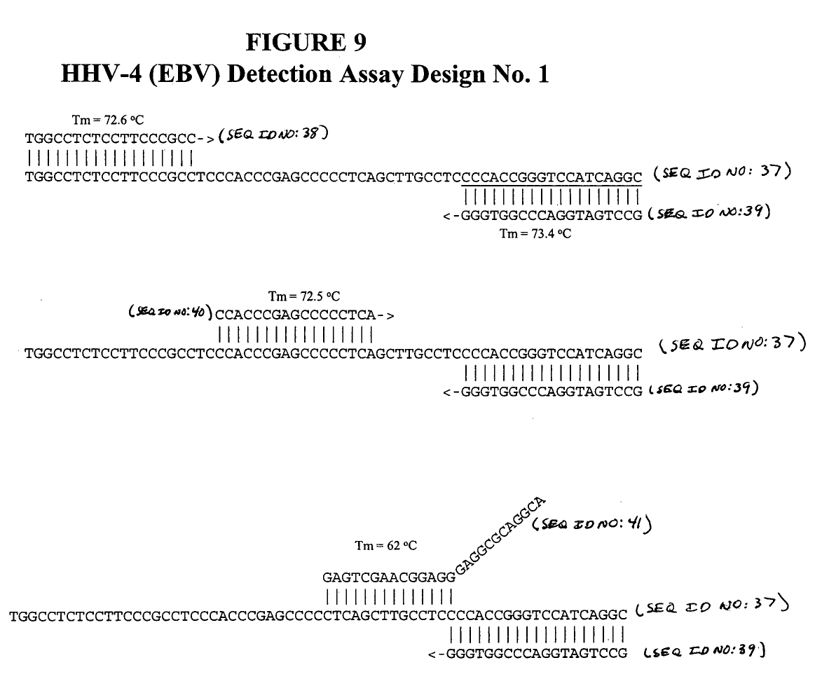

FIG. 9 shows a detection assay design for detecting HHV-4 (also known as Epstein-Barr Virus or EBV). In this Figure, SEQ ID NO:37 is a selected portion of the BamHI-W region (nt 33505-36576) which is to be PCR amplified, SEQ ID NOs:38 and 40 are forward primers (Tm=72.6C and 72.5 respectively), SEQ ID NO:39 is a reverse primer (Tm=73.4) which also doubles as the INVADER oligonucleotide, and SEQ ID NO:41 is the primary probe (Tm=62.0C).

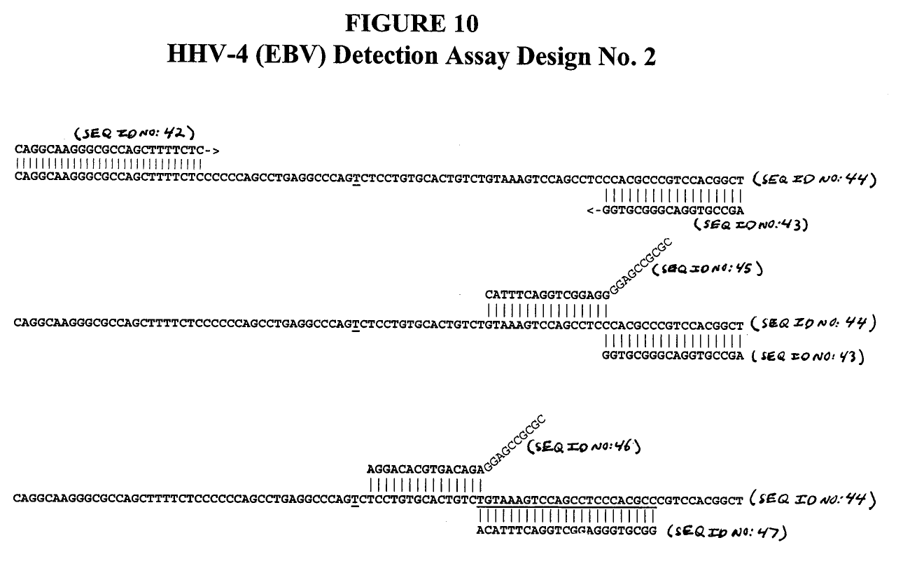

FIG. 10 shows an additional detection assay design for detecting HHV-4. In this Figure, SEQ ID NO:44 is a selected portion of the BamHI-W region which is to be PCR amplified, SEQ ID NO:42 is a forward primer, SEQ ID NOs:43 and 47 are reverse primers which also double as INVADER oligonucleotides, and SEQ ID NOs:45 and 46 are primary probes.

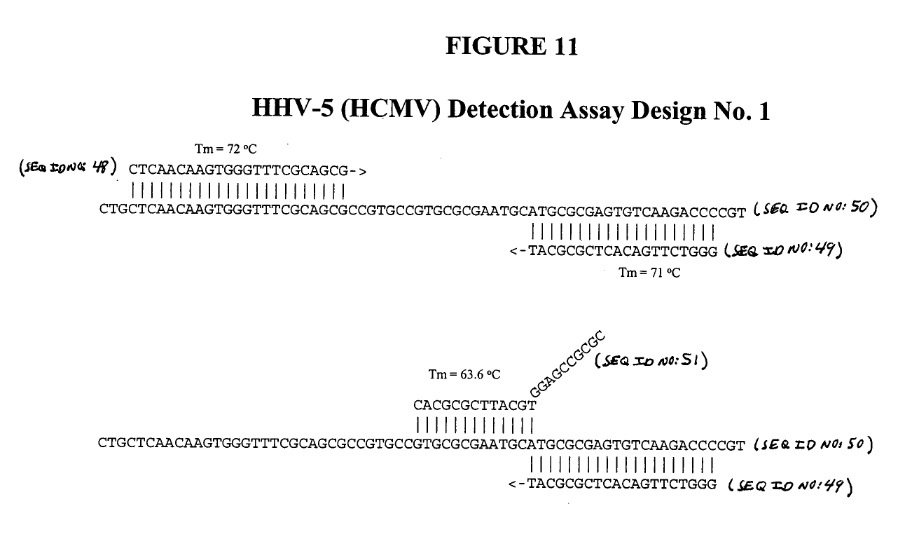

FIG. 11 shows a detection assay design for detecting HHV-5 (also known as human cytomegalovirus or HCMV). In this Figure, SEQ ID NO:50 is a selected portion of the HHV-5 DNA polymerase gene, a portion of which is to be PCR amplified, SEQ ID NO:48 is a forward primer (Tm=72.0C), SEQ ID NO:49 is a reverse primer (Tm=71.0C) which also doubles as the INVADER oligonucleotide, and SEQ ID NO:51 is the primary probe (Tm=63.6C).

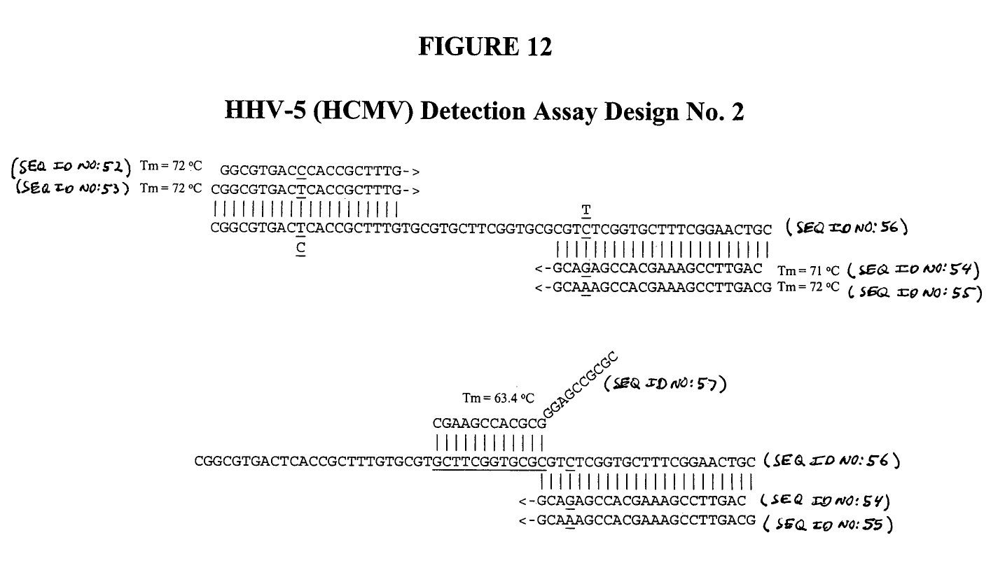

FIG. 12 shows an additional detection assay design for detecting HHV-5. In this Figure, SEQ ID NO:56 is a selected portion of the HHV-5 DNA polymerase gene at least a portion of which is to be PCR amplified, SEQ ID NOs:52 and 53 are forward primers (both with a Tm of 72C), SEQ ID NOs:54 and 55 are reverse primers (Tm=71C and 72C respectively) both of which double as INVADER oligonucleotides, and SEQ ID NO:57 is the primary probe (Tm=63.4C).

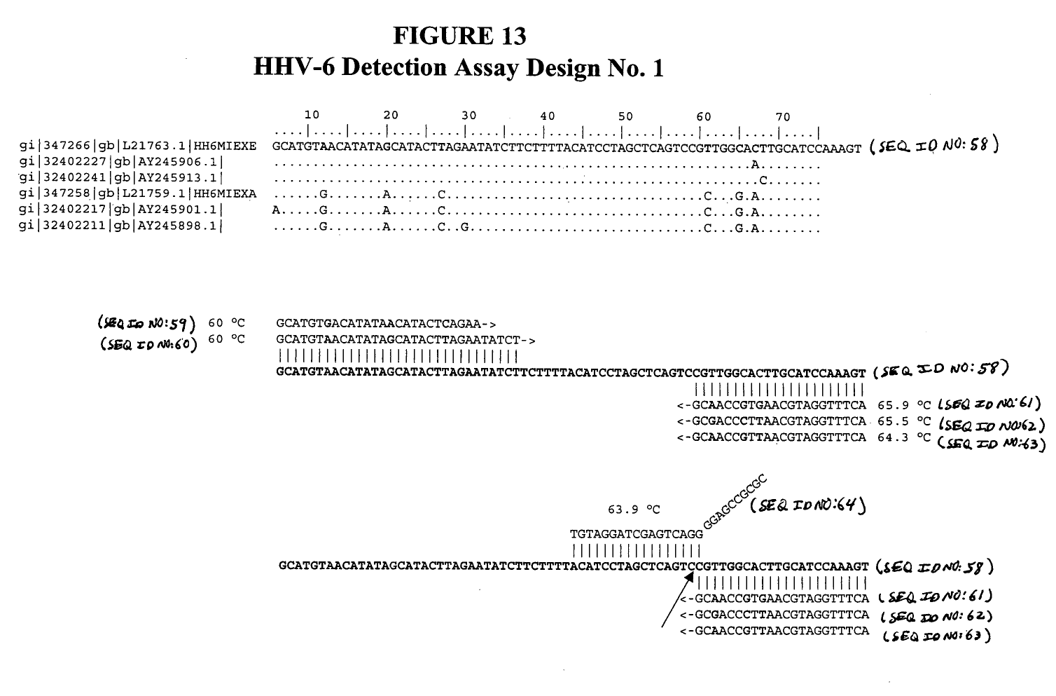

FIG. 13 shows a detection assay design for detecting HHV-6 (human Herpes virus 6). In this Figure, SEQ ID NO:58 is a selected portion of the HHV-6 U90 gene which is to be PCR amplified, SEQ ID NOs:59 and 60 are forward primers (both Tm=60.0C), SEQ ID NOs:61, 62, and 63 are reverse primers (with Tm's of 65.9C, 65.5C, and 64.3C respectively) all of which double as INVADER oligonucleotides, and SEQ ID NO:64 is the primary probe (Tm=63.9C). This figures also shows an alignment of some of the HHV-6 variants, visually depicting how one could select conserved regions for the primary probe, as well as the PCR primers.

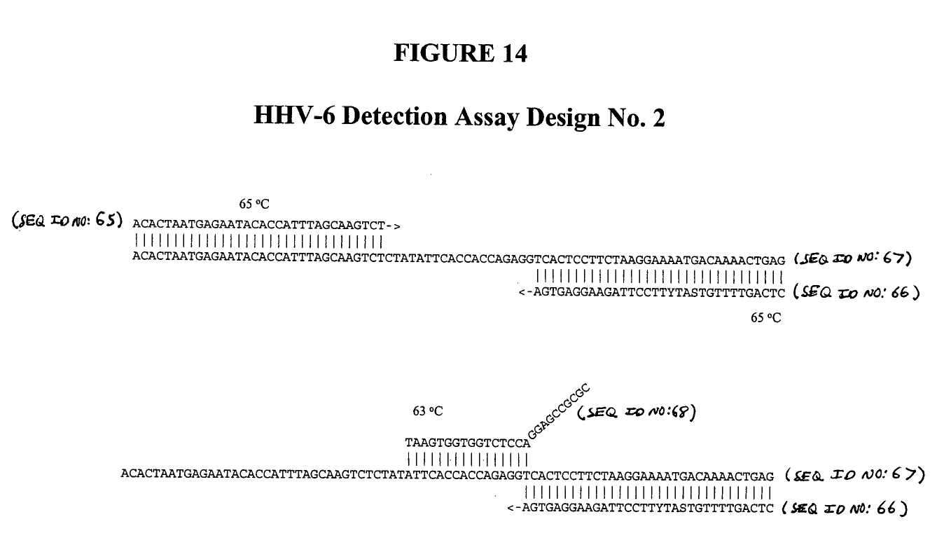

FIG. 14 shows an additional detection assay design for detecting HHV-6. In this Figure, SEQ ID NO:67 is a selected portion of the HHV-6 U90 gene which is to be PCR amplified, SEQ ID NO:65 is a forward primer (Tm=65.0C), SEQ ID NO:66 is a reverse primer (Tm=65.0C) which also doubles as the INVADER oligonucleotide, and SEQ ID NO:68 is the primary probe (Tm=63.0C).

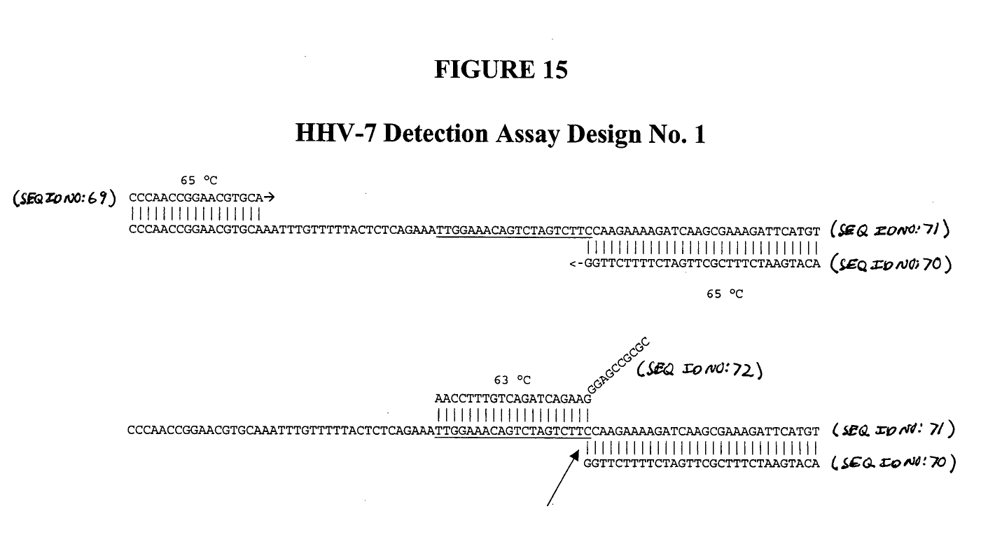

FIG. 15 shows a detection assay design for detecting HHV-7 (Human Herpes Virus 7). In this Figure, SEQ ID NO:71 is a selected portion of the HHV-7 MYG gH gene which is to be PCR amplified, SEQ ID NO:69 is a forward primer (Tm=65.0C), SEQ ID NO:70 is a reverse primer (Tm=65.0C) which also doubles as the INVADER oligonucleotide, and SEQ ID NO:72 is the primary probe (Tm=63.0C).

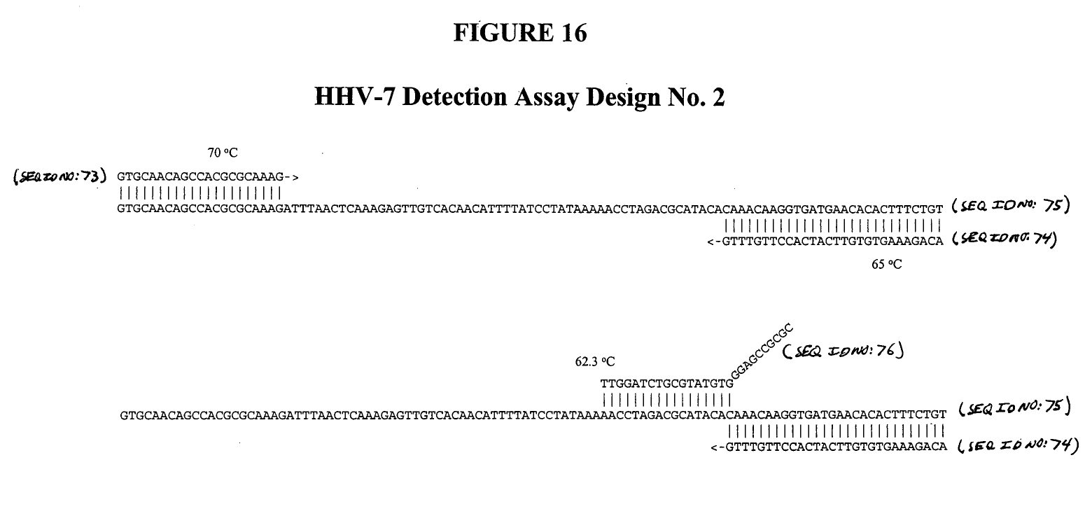

FIG. 16 shows an additional detection assay design for detecting HHV-7. In this Figure, SEQ ID NO:75 is a selected portion of the HHV-7 MYG gH gene which is to be PCR amplified, SEQ ID NO:73 is a forward primer (Tm=70.0C), SEQ ID NO:74 is a reverse primer (Tm=65.0C) which also doubles as the INVADER oligonucleotide, and SEQ ID NO:76 is the primary probe (Tm=62.3C).

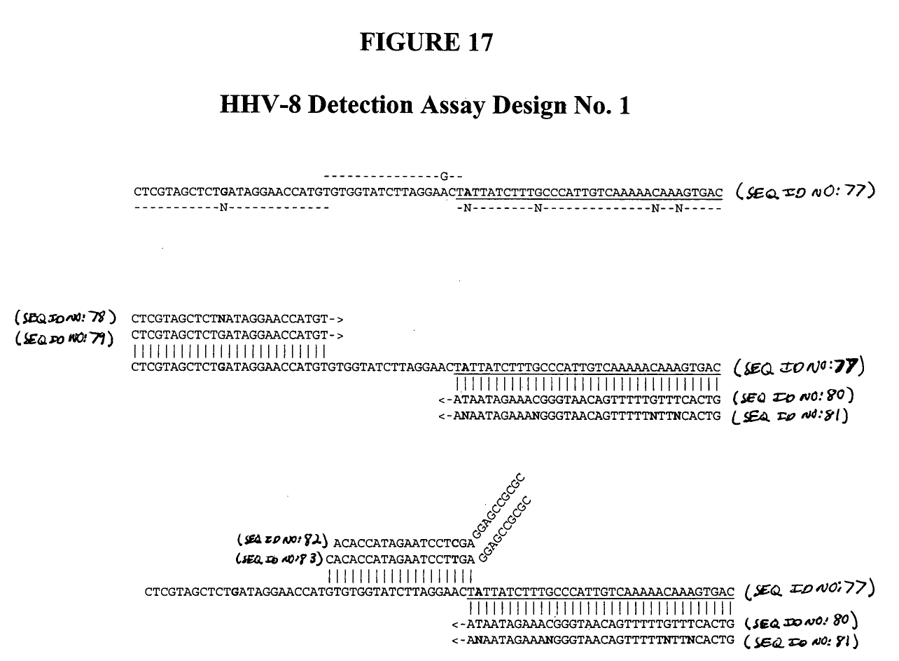

FIG. 17 shows a detection assay design for detecting HHV-8 (also known as Kaposi Sarcoma Herpesvirus or KSHV). In this Figure, SEQ ID NO:77 is a selected portion of the HHV-8 glycoprotein K1 gene which is to be PCR amplified, SEQ ID NOs:78 and 79 are forward primers, SEQ ID NOs:80 and 81 are reverse primers, both of which also double as INVADER oligonucleotides, and SEQ ID NOs:82 and 83 are primary probes.

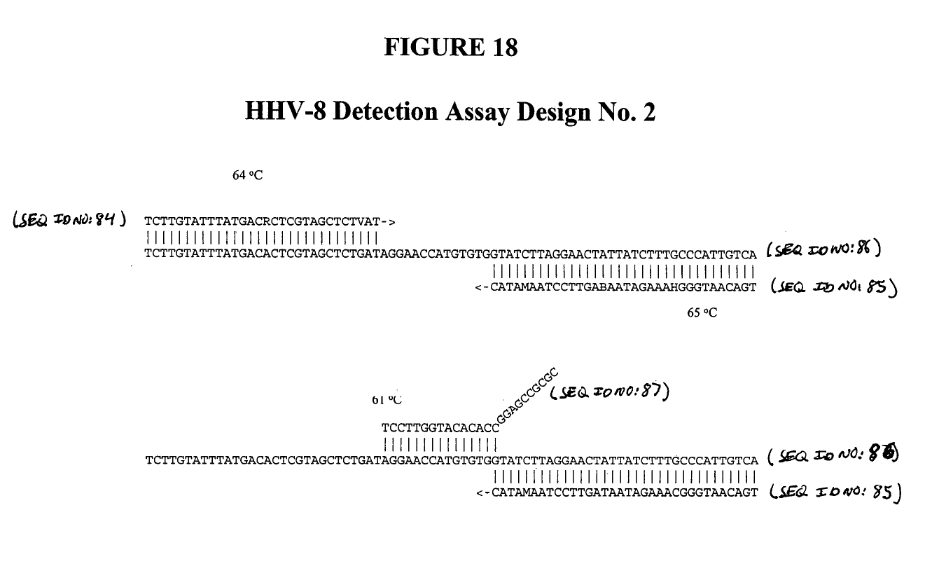

FIG. 18 shows a detection assay design for detecting HHV-8. In this Figure, SEQ ID NO:86 is a selected portion of the HHV-8 glycoprotein KI gene which is to be PCR amplified, SEQ ID NO:84 is a forward primer (Tm=64.0C), SEQ ID NO:85 is a reverse primer (Tm=65.0C) which also doubles as the INVADER oligonucleotide, and SEQ ID NO:87 is the primary probe (Tm=61.0C).

The above assays find use to detect the various sub-types of human herpes virus. These assays find use alone or together in order to detect and differentiate among the various subtypes.

### Example 2

### Detection of HCMV (HHV-5) Nucleic Acid

The following experimental example describes the use of an invasive cleavage assay to detect HHV-5, also called human cytomegalovirus (HCMV), DNA. In this Example, HCMV DNA was detected from a prequantitated viral DNA standard using PCR amplification followed by the INVADER assay. The detection assay employed is shown in FIG. 11. The forward primer was SEQ ID NO:48 and the reverse primer was SEQ ID NO:49. The reverse PCR primer also served as the invasive oligonucleotide in the INVADER assay. The probe oligonucleotide was SEQ ID NO:51, and the FRET-labeled oligonucleotide (not shown in FIG. 11) was 5′-YTCTXAAGCCGGTTTTCCGGCTGAGACCTCGGCGCG-3′, where Y is FAM and X is Z28 (SEQ ID NO:18).

The reaction conditions were as follows: forward and reverse PCR primer at 0.4 uM each, probe oligonucleotide at 0.67 uM, FRET oligonucleotide at 0.33 uM, MOPS buffer at 10 mM, MgCl2 at 7.5 mM, dNTPs at 25 uM, native Taq polymerase at 0.3 units per reaction, Cleavase VIII at 100 ng per reaction, and the balance H2O in a total reaction volume of 15 uL. 5 uL of this reaction volume was comprised of diluted pre-quantitated HCMV template DNA ranging from 2 to 2500 copies per reaction. The reaction temperature conditions were as follows: 26 cycles of 95° C. for 30 sec, 64° C. for 30 sec, and 72° C. for 2 min; 99° C. for 10 m

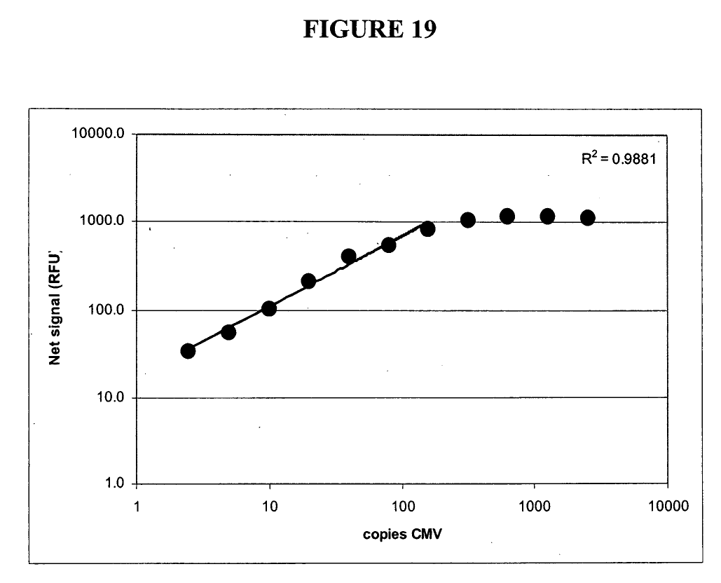

The results are presented in FIG. 19, which shows that the INVADER assay can detect HCMV DNA. As shown in this Figure, the assay detected HCMV DNA from 2 to 625-1250 copies per reaction in a concentration-dependent fashion.

### Example 3

### Detection of HCMV (HHV-5) Nucleic Acid From a Biological Specimen Sample

The following Example describes the detection of HCMV (HHV-5) nucleic acid from a biological specimen sample using an invasive cleavage assay, in this case human plasma. Nucleic acid was purified from the plasma using the Purigene kit (Gentra Systems, Plymouth, Minn.). The purified DNA was then subjected to PCR amplification and detection using the INVADER assay.

The oligonucleotides as described in Example 2 and as shown in FIG. 11 were used in this Example. The reaction conditions were as follows: forward and reverse PCR primer at 0.4 uM each, probe oligonucleotide at 0.67 uM, FRET oligonucleotide at 0.33 uM, MOPS buffer at 10 mM, MgCl2 at 7.5 mM, dNTPs at 25 uM, native Taq polymerase at 0.3 units per reaction, Cleavase VIII at 100 ng per reaction, and the balance H20 in a total reaction volume of 15 uL. 5 uL of this reaction volume was comprised of either a diluted pre-quantitated HCMV template DNA ranging from 0 to 500 copies per reaction, or a DNA sample derived from a HCMV-positive or HCMV-negative plasma sample. The reaction temperature conditions were as follows: 26 cycles of 95° C. for 30 sec, and 72° C. for 1 min; 99° C. for 10 min; and 63° C. for 20 min.

As shown in Table 2, the results of this experiment demonstrated that the INVADER assay can detect HCMV DNA from a biological specimen sample. “Average” in this table refers to average raw fluorescence counts of the INVADER assay signal from three replicate reactions.

TABLE 2

| viral DNA | Average |
|---|---|
| copies/rxn | 843 |
| 500 | 843 |
| 167 | 1000 |
| 56 | 644 |
| 19 | 182 |
| 6 | 187 |
| 2 | 53 |
| 1 | 53 |
| 0 | 55 |
| Positive plasma | |
| 50000 | 1320 |
| 12500 | 1298 |
| 3125 | 1207 |
| 781 | 498 |
| 195 | 65 |
| 49 | 238 |
| 12 | 59 |
| negative plasma | 61 |
| Positive plasma | |
| 50000 | 1323 |
| 12500 | 1339 |
| 3125 | 1298 |
| 781 | 794 |
| 195 | 170 |
| 49 | 64 |
| 12 | 63 |
| negative plasma | 62 |

All publications and patents mentioned in the above specification are herein incorporated by reference as if expressly set forth herein. Various modifications and variations of the described assays of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method of detecting the presence or absence of at least one human herpes virus subtype in a sample comprising;
a) contacting said sample with first and second oligonucleotides, wherein said first and second oligonucleotides are configured to form an invasive cleavage structure with a target sequence, wherein said target sequence comprises human herpes virus sequence, and
b) detecting the presence or absence of said at least one human herpes virus subtype in said sample.

2. The method of claim 1, wherein said detecting comprises observing a signal generated by cleavage of said first oligonucleotide, thereby identifying the presence of said at least one human herpes virus subtype in said sample.

3. The method of claim 1, wherein said at least one human herpes virus subtype is selected from the group consisting of: HHV-1, HHV-2, HHV-3, HHV-4, HHV-5, HHV-6, HHV-7, and HHV-8.

4. The method of claim 1, wherein said first oligonucleotide comprises a 5′ portion and a 3′ portion, wherein said 3′ portion is configured to hybridize to said target sequence, and wherein said 5′ portion is configured to not hybridize to said target sequence.

5. The method of claim 1, wherein said second oligonucleotide comprises a 5′ portion and a 3′ portion, wherein said 5′ portion is configured to hybridize to said target sequence, and wherein said 3′ portion is configured to not hybridize to said target sequence.

6. The method of claim 1, wherein said second oligonucleotide also serves as a first PCR primer configured to amplify said target sequence with a second PCR primer.

7. The method of claim 6, wherein said second oligonucleotide, which also serves as said first PCR primer, is selected from the group consisting of: SEQ ID NO:4, SEQ ID NO:8, SEQ ID NO:13, SEQ ID NO:19, SEQ ID NO:22, SEQ ID NO:26, SEQ ID NO:30, SEQ ID NO:35, SEQ ID NO:39, SEQ ID NO:43, SEQ ID NO:47, SEQ ID NO:49, SEQ ID NO:54, SEQ ID NO:55, SEQ ID NO:61, SEQ ID NO:62, SEQ ID NO:63, SEQ ID NO:66, SEQ ID NO:70, SEQ ID NO:74, SEQ ID NO:80, SEQ ID NO:81, and SEQ ID NO:85.

8. The method of claim 6, further comprising contacting said sample with said second PCR primer.

9. The method of claim 6, wherein said second PCR primer is selected from the group consisting of: SEQ ID NO:3, SEQ ID NO:9, SEQ ID NO:11, SEQ ID NO:12, SEQ ID NO:17, SEQ ID NO:23, SEQ ID NO:27, SEQ ID NO:31, SEQ ID NO:34, SEQ ID NO:38, SEQ ID NO:40, SEQ ID NO:42, SEQ ID NO:48, SEQ ID NO:52, SEQ ID NO:53, SEQ ID NO:59, SEQ ID NO:60, SEQ ID NO:65, SEQ ID NO:69, SEQ ID NO:73, SEQ ID NO:78, SEQ ID NO:79, and SEQ ID NO:84.

10. The method of claim 1, wherein said first oligonucleotide is selected from the group consisting of: SEQ ID NO:5, SEQ ID NO:10, SEQ ID NO:14, SEQ ID NO:20, SEQ ID NO:24, SEQ ID NO:28, SEQ ID NO:32, SEQ ID NO:36, SEQ ID NO:41, SEQ ID NO:45, SEQ ID NO:46, SEQ ID NO:51, SEQ ID NO:57, SEQ ID NO:64, SEQ ID NO:68, SEQ ID NO:72, SEQ ID NO:76, SEQ ID NO:82, SEQ ID NO:83, and SEQ ID NO:87.

11. The method of claim 1, further comprising contacting said sample with a FRET cassette.

12. The method of claim 1, wherein said target sequence comprises a PCR amplified product.

13. The method of claim 1, wherein said target sequence comprises genomic herpes virus nucleic acid.

14. The method of claim 1, wherein said first or second oligonucleotide comprises one or more mismatches with said target sequence.

15. The method of claim 1, wherein said first or second oligonucleotide does not hybridize to human genomic DNA under stringent conditions.

16. The method of claim 1, wherein said first and second oligonucleotides are configured such that a stable duplex between said first and second oligonucleotides and said target sequence is not formed.

17. A kit for detecting the presence or absence of at least one human herpes virus subtype in a sample comprising;
a) first and second oligonucleotides configured to form an invasive cleavage structure with a target sequence, wherein said target sequence comprises human herpes virus sequences, and
b) a cleavage agent, wherein said cleavage agent is capable of cleaving said first oligonucleotide when said cleavage structure is formed.

18. The kit of claim 17, wherein said first oligonucleotide comprises a 5′ portion and a 3′ portion, wherein said 3′ portion is configured to hybridize to said target sequence, and wherein said 5′ portion is configured to not hybridize to said target sequence.

19. The kit of claim 17, wherein said second oligonucleotide comprises a 5′ portion and a 3′ portion, wherein said 5′ portion is configured to hybridize to said target sequence, and wherein said 3′ portion is configured to not hybridize to said target sequence.

20. The kit of claim 17, wherein said second oligonucleotide also serves as a first PCR primer configured to PCR amplify said target sequence with a second PCR primer.


SEQUENCE LISTING (87 sequences)

SEQ ID NO: 1 - DNA, 100 bp, human herpesvirus 1
gttatttaccctgtttcgggcccccgagttgctggcccccaacggcgacctgtataacgtgtttgcctgggctttggacgtcttggccaaacgcctccgt

SEQ ID NO: 2 - DNA, 100 bp, human herpesvirus 2
gctgtttgccctgttccgcgttcccgagctgctggcccccaacggggacttgtaccacatttttgcctgggtcttggacgtcttggccgaccgcctcctt

SEQ ID NO: 3 - DNA, 29 bp, Artificial Sequence
  Synthetic
gttatttaccctgtttcgggcccccgagt

SEQ ID NO: 4 - DNA, 26 bp, Artificial Sequence
  Synthetic
ggaggcgtttgrccaagacgtccaaa

SEQ ID NO: 5 - DNA, 26 bp, Artificial Sequence
  Synthetic
acaaacggacccgagaggcgcaggca

SEQ ID NO: 6 - DNA, 96 bp, human herpesvirus 1
gtcttggccaaacgcctccgtcccatgcatgtctttatcctggattacgaccaatcgcccgccggctgccgggacgccctgctgcaacttacctcc

SEQ ID NO: 7 - DNA, 96 bp, human herpesvirus 2
gtcttggccgaccgcctccttccgatgcatctatttgtcctggattacgatcagtcgcccgtcgggtgtcgagacgccctgttgcgcctcaccgcc

SEQ ID NO: 8 - DNA, 24 bp, Artificial Sequence
  Synthetic
gcctccgtcccatgcatgtcttta

SEQ ID NO: 9 - DNA, 21 bp, Artificial Sequence
  Synthetic
cctgcgggacgacgttgaatg

SEQ ID NO: 10 - DNA, 28 bp, Artificial Sequence
  Synthetic
acggacgcggagatcctggattacgacc

SEQ ID NO: 11 - DNA, 27 bp, Artificial Sequence
  Synthetic
ctgctggcccccaacggggacttgtac

SEQ ID NO: 12 - DNA, 21 bp, Artificial Sequence
  Synthetic
ccctgttccgcgttcccgagc

SEQ ID NO: 13 - DNA, 23 bp, Artificial Sequence
  Synthetic
ggcggtcggccaagacgtccaag

SEQ ID NO: 14 - DNA, 28 bp, Artificial Sequence
  Synthetic
acggacgcggaggacccaggcaaaaatg

SEQ ID NO: 15 - DNA, 90 bp, human herpesvirus 1
acgtgtttgcctgggctttggacgtcttggccaaacgcctccgtcccatgcatgtctttatcctggattacgaccaatcgcccgccggct

SEQ ID NO: 16 - DNA, 90 bp, human herpesvirus 2
acatttttgcctgggtcttggacgtcttggccgaccgcctccttccgatgcatctatttgtcctggattacgatcagtcgcccgtcgggt

SEQ ID NO: 17 - DNA, 20 bp, Artificial Sequence
  Synthetic
ggtcttggacgtcttggccg

SEQ ID NO: 18 - DNA, 34 bp, Artificial Sequence
  Synthetic
tctaagccggttttccggctgagacctcggcgcg

SEQ ID NO: 19 - DNA, 37 bp, Artificial Sequence
  Synthetic
gggcgactgatcgtaatccaggacaaatagatgcatc

SEQ ID NO: 20 - DNA, 25 bp, Artificial Sequence
  Synthetic
acggacgcggagcggaaggaggcgg

SEQ ID NO: 21 - DNA, 90 bp, Human herpesvirus 3
acccggtctgctcatctgggcgatggtgatgaaatcagagaagctatacacaagtcccaggacgccgaaacaaaacccacgttttacgtc

SEQ ID NO: 22 - DNA, 23 bp, Artificial Sequence
  Synthetic
gctcatctgggcgatggtgatga

SEQ ID NO: 23 - DNA, 22 bp, Artificial Sequence
  Synthetic
ggttttgtttcggcgtcctggg

SEQ ID NO: 24 - DNA, 34 bp, Artificial Sequence
  Synthetic
acggacgcggagaaatcagagaagctatacacaa

SEQ ID NO: 25 - DNA, 96 bp, Human herpesvirus 3
aaatataattctgtgggatccaaagcatggcatactaccaatgacacgtacatggttgccggaacccccggaacatataggacgggcacgtcggtg

SEQ ID NO: 26 - DNA, 28 bp, Artificial Sequence
  Synthetic
ctgtgggatccaaagcatggcatactac

SEQ ID NO: 27 - DNA, 23 bp, Artificial Sequence
  Synthetic
ctatatgttccgggggttccggc

SEQ ID NO: 28 - DNA, 30 bp, Artificial Sequence
  Synthetic
acggacgcggagccaatgacacgtacatgg

SEQ ID NO: 29 - DNA, 91 bp, Human herpesvirus 3
ctaataaatcttacgtaacaccaacccctgcgactcgctctatcggacatatgtctgctcttctacgagaatattccgaccgtaatatgtc

SEQ ID NO: 30 - DNA, 24 bp, Artificial Sequence
  Synthetic
cttacgtaacaccaacccctgcga

SEQ ID NO: 31 - DNA, 29 bp, Artificial Sequence
  Synthetic
acggtcggaatattctcgtagaagagcag

SEQ ID NO: 32 - DNA, 28 bp, Artificial Sequence
  Synthetic
acggacgcggagactcgctctatcggac

SEQ ID NO: 33 - DNA, 91 bp, Human herpesvirus 3
caaagcagaacatcgagcaccgtttcctgctggacgttttggatttcttagtcaccctgtgacacccgacgtgagcttctttgacagttcg

SEQ ID NO: 34 - DNA, 21 bp, Artificial Sequence
  Synthetic
agcaccgtttcctgctggacg

SEQ ID NO: 35 - DNA, 24 bp, Artificial Sequence
  Synthetic
ctgtcaaagaagctcacgtcgggt

SEQ ID NO: 36 - DNA, 29 bp, Artificial Sequence
  Synthetic
acggacgcggagtgtcacagggtgactaa

SEQ ID NO: 37 - DNA, 65 bp, Human herpesvirus 4
tggcctctccttcccgcctcccacccgagccccctcagcttgcctccccaccgggtccatcaggc

SEQ ID NO: 38 - DNA, 18 bp, Artificial Sequence
  Synthetic
tggcctctccttcccgcc

SEQ ID NO: 39 - DNA, 19 bp, Artificial Sequence
  Synthetic
gggtggcccaggtagtccg

SEQ ID NO: 40 - DNA, 17 bp, Artificial Sequence
  Synthetic
ccacccgagccccctca

SEQ ID NO: 41 - DNA, 26 bp, Artificial Sequence
  Synthetic
gagtcgaacggagggaggcgcaggca

SEQ ID NO: 42 - DNA, 24 bp, Artificial Sequence
  Synthetic
caggcaagggcgccagcttttctc

SEQ ID NO: 43 - DNA, 18 bp, Artificial Sequence
  Synthetic
ggtgcgggcaggtgccga

SEQ ID NO: 44 - DNA, 93 bp, Human herpesvirus 4
caggcaagggcgccagcttttctccccccagcctgaggcccagtctcctgtgcactgtctgtaaagtccagcctcccacgcccgtccacggct

SEQ ID NO: 45 - DNA, 26 bp, Artificial Sequence
  Synthetic
catttcaggtcggaggggagccgcgc

SEQ ID NO: 46 - DNA, 25 bp, Artificial Sequence
  Synthetic
aggacacgtgacagaggagccgcgc

SEQ ID NO: 47 - DNA, 23 bp, Artificial Sequence
  Synthetic
acatttcaggtcggagggtgcgg

SEQ ID NO: 48 - DNA, 23 bp, Artificial Sequence
  Synthetic
ctcaacaagtgggtttcgcagcg

SEQ ID NO: 49 - DNA, 20 bp, Artificial Sequence
  Synthetic
tacgcgctcacagttctggg

SEQ ID NO: 50 - DNA, 68 bp, Human herpesvirus 5
ctgctcaacaagtgggtttcgcagcgccgtgccgtgcgcgaatgcatgcgcgagtgtcaagaccccgt

SEQ ID NO: 51 - DNA, 23 bp, Artificial Sequence
  Synthetic
cacgcgcttacgtggagccgcgc

SEQ ID NO: 52 - DNA, 19 bp, Artificial Sequence
  Synthetic
ggcgtgacccaccgctttg

SEQ ID NO: 53 - DNA, 20 bp, Artificial Sequence
  Synthetic
cggcgtgactcaccgctttg

SEQ ID NO: 54 - DNA, 22 bp, Artificial Sequence
  Synthetic
gcagagccacgaaagccttgac

SEQ ID NO: 55 - DNA, 23 bp, Artificial Sequence
  Synthetic
gcaaagccacgaaagccttgacg

SEQ ID NO: 56 - DNA, 59 bp, Human herpesvirus 5
cggcgtgactcaccgctttgtgcgtgcttcggtgcgcgtctcggtgctttcggaactgc

SEQ ID NO: 57 - DNA, 22 bp, Artificial Sequence
  Synthetic
cgaagccacgcgggagccgcgc

SEQ ID NO: 58 - DNA, 75 bp, Human herpesvirus 6
gcatgtaacatatagcatacttagaatatcttcttttacatcctagctcagtccgttggcacttgcatccaaagt

SEQ ID NO: 59 - DNA, 26 bp, Artificial Sequence
  Synthetic
gcatgtgacatataacatactcagaa

SEQ ID NO: 60 - DNA, 31 bp, Artificial Sequence
  Synthetic
gcatgtaacatatagcatacttagaatatct

SEQ ID NO: 61 - DNA, 22 bp, Artificial Sequence
  Synthetic
gcaaccgtgaacgtaggtttca

SEQ ID NO: 62 - DNA, 22 bp, Artificial Sequence
  Synthetic
gcgacccttaacgtaggtttca

SEQ ID NO: 63 - DNA, 22 bp, Artificial Sequence
  Synthetic
gcaaccgttaacgtaggtttca

SEQ ID NO: 64 - DNA, 27 bp, Artificial Sequence
  Synthetic
tgtaggatcgagtcaggggagccgcgc

SEQ ID NO: 65 - DNA, 32 bp, Artificial Sequence
  Synthetic
acactaatgagaatacaccatttagcaagtct

SEQ ID NO: 66 - DNA, 32 bp, Artificial Sequence
  Synthetic
agtgaggaagattccttytastgttttgactc

SEQ ID NO: 67 - DNA, 83 bp, Human herpesvirus 6
acactaatgagaatacaccatttagcaagtctctatattcaccaccagaggtcactccttctaaggaaaatgacaaaactgag

SEQ ID NO: 68 - DNA, 26 bp, Artificial Sequence
  Synthetic
taagtggtggtctccaggagccgcgc

SEQ ID NO: 69 - DNA, 17 bp, Artificial Sequence
  Synthetic
cccaaccggaacgtgca

SEQ ID NO: 70 - DNA, 30 bp, Artificial Sequence
  Synthetic
ggttcttttctagttcgctttctaagtaca

SEQ ID NO: 71 - DNA, 88 bp, Human herpesvirus 7
cccaaccggaacgtgcaaatttgtttttactctcagaaattggaaacagtctagtcttccaagaaaagatcaagcgaaagattcatgt

SEQ ID NO: 72 - DNA, 30 bp, Artificial Sequence
  Synthetic
aacctttgtcagatcagaagggagccgcgc

SEQ ID NO: 73 - DNA, 21 bp, Artificial Sequence
  Synthetic
gtgcaacagccacgcgcaaag

SEQ ID NO: 74 - DNA, 28 bp, Artificial Sequence
  Synthetic
gtttgttccactacttgtgtgaaagaca

SEQ ID NO: 75 - DNA, 105 bp, Human herpesvirus 7
gtgcaacagccacgcgcaaagatttaactcaaagagttgtcacaacattttatcctataaaaacctagacgcatacacaaacaaggtgatgaacacactttctgt

SEQ ID NO: 76 - DNA, 27 bp, Artificial Sequence
  Synthetic
ttggatctgcgtatgtgggagccgcgc

SEQ ID NO: 77 - DNA, 75 bp, Human herpesvirus 8
ctcgtagctctgataggaaccatgtgtggtatcttaggaactattatctttgcccattgtcaaaaacaaagtgac

SEQ ID NO: 78 - DNA, 25 bp, Artificial Sequence
  Synthetic
ctcgtagctctnataggaaccatgt

SEQ ID NO: 79 - DNA, 25 bp, Artificial Sequence
  Synthetic
ctcgtagctctgataggaaccatgt

SEQ ID NO: 80 - DNA, 34 bp, Artificial Sequence
  Synthetic
ataatagaaacgggtaacagtttttgtttcactg

SEQ ID NO: 81 - DNA, 34 bp, Artificial Sequence
  Synthetic
anaatagaaangggtaacagtttttnttncactg

SEQ ID NO: 82 - DNA, 28 bp, Artificial Sequence
  Synthetic
acaccatagaatcctcgaggagccgcgc

SEQ ID NO: 83 - DNA, 29 bp, Artificial Sequence
  Synthetic
cacaccatagaatccttgaggagccgcgc

SEQ ID NO: 84 - DNA, 30 bp, Artificial Sequence
  Synthetic
tcttgtatttatgacrctcgtagctctvat

SEQ ID NO: 85 - DNA, 34 bp, Artificial Sequence
  Synthetic
catamaatccttgabaatagaaahgggtaacagt

SEQ ID NO: 86 - DNA, 78 bp, Human herpesvirus 8
tcttgtatttatgacactcgtagctctgataggaaccatgtgtggtatcttaggaactattatctttgcccattgtca

SEQ ID NO: 87 - DNA, 25 bp, Artificial Sequence
  Synthetic
tccttggtacacaccggagccgcgc